(19) United States
(12) Patent Application Publication
(10) Pub. No.: US 2025/0221923 A1
(43) Pub. Date: Jul. 10, 2025

(54) BOOSTING OSMOTIC BLOOD-ORGAN BARRIER OPENING FOR IMPROVED DELIVERY OF THERAPEUTICS TO ORGANS

(71) Applicant: University of Maryland, Baltimore, Baltimore, MD (US)

(72) Inventors: Miroslaw JANOWSKI, Baltimore, MD (US); Piotr WALCZAK, Baltimore, MD (US); Guanda QIAO, Baltimore, MD (US); Chengyan CHU, Baltimore, MD (US); David GULISASHVILI, Clarksville, MD (US)

(21) Appl. No.: 19/008,900

(22) Filed: Jan. 3, 2025

Related U.S. Application Data
(60) Provisional application No. 63/617,652, filed on Jan. 4, 2024.
(60) Provisional application No. 63/691,377, filed on Sep. 6, 2024.

(51) Int. Cl. A61K 9/00; A61K 45/06; A61K 47/02; A61K 47/10; A61K 47/26

(52) U.S. Cl. CPC A61K 9/0004; A61K 9/0019; A61K 45/06; A61K 47/02; A61K 47/10; A61K 47/26

(57) ABSTRACT

Osmotic blood-tissue barrier opening offers a promising approach to surmounting challenges posed by the blood-brain barrier in the treatment of brain cancer and neurological diseases. By enhancing permeability of any blood-organ barrier, enabling precise targeting, reducing toxicity, being less invasive, and potentially accommodating larger therapeutic molecules, osmotic blood-organ barrier opening represents a valuable strategy in the fight against diseases such as brain cancer and neurological diseases.

### CROSS REFERENCE TO RELATED APPLICATIONS

This application is filed under the provisions of 35 U.S.C. § 111 (a) and claims priority to U.S. Provisional Patent Application No. 63/617,652 filed on Jan. 4, 2024 in the name of Miroslaw JANOSKI, et al., and entitled “Boosting Osmotic Blood-Brain Barrier Opening,” and U.S. Provisional Patent Application No. 63/691,377 filed on Sep. 6, 2024 in the name of Miroslaw JANOSKI, et al., and entitled “Boosting Osmotic Blood-Brain Barrier Opening,” both of which are hereby incorporated by reference herein in their entirety.

### STATEMENT OF FEDERALLY SPONSORED RESEARCH

This invention was made with government support under Grant Number NS120929 awarded by the National Institutes of Health. The government has certain rights in the invention.

### FIELD

The present invention relates to methods of the osmotic opening of blood-organ barriers, including osmotic blood-brain barrier opening (OBBBO), to improve the delivery of therapeutic agents in the treatment of cancer, e.g., brain cancer, head and neck cancer, inflammatory diseases, genetic diseases, neurological diseases, mental diseases, and virtually disease of any organ that could benefit from local precision delivery or the higher local concentration of the therapeutic agents, than is achievable using current methods.

### DESCRIPTION OF THE RELATED ART

Neurodegenerative diseases, such as Alzheimer's and Parkinson's disease, are an increasing burden on our ageing society because there are currently no effective treatments for these disabling conditions. Treatment as well as early diagnosis of these and other diseases that originate in the brain remain challenging because the majority of suitable therapeutic molecules and diagnostics cannot penetrate the tight and highly restrictive blood-brain barrier (BBB) [Abbott, 2013]. The BBB constitutes a physical and physiological barricade that is formed by brain endothelial cells (BECs) that line the blood vessels and connect with each other through tight junctions [Abbott, 2013]. The tight junctions formed between the BECs are essential for the integrity of the BBB and prevent the paracellular transport of hydrophilic molecules larger than 500 daltons (Da). Because brain endothelial cells exhibit very low pinocytosis rates [Abbott, 2013], transcellular transport of larger molecules is limited to the highly specific receptor mediated transcytosis (RMT) pathway, and the passive, charge-based adsorption mediated transcytosis [Abbott, 2013; Pardridge, 2002].

While all these characteristics protect the brain from pathogens and toxins, they equally prevent the entry of most therapeutics. In fact, less than 5% of small molecule therapeutics and virtually none of the larger therapeutics can cross the BBB in pharmacologically relevant concentrations (i.e., sufficient to engage a central nervous system (CNS) target and elicit pharmacologic/therapeutic response) unless they are specifically “ferried.” that is, coupled to a transporter molecule. Due to the lack of effective carriers to transport molecules across the BBB, numerous drugs against neurodegenerative, neurooncological and psychiatric diseases have been shelved or eliminated from further development as they cannot be delivered to the brain in sufficient amount.

Different approaches to deliver larger molecules into the brain have been explored. For example, the integrity of the BBB may be disrupted, resulting in a leaky BBB, which in turn allows for unrestricted, paracellular entry of larger molecules into the brain. A more localized disruption of the BBB is possible through the application of focused ultrasound [Nhan, 2013]. However, the periods during which the BBB is disrupted are sufficient to alter brain homeostasis and allow harmful chemicals, toxins and pathogens to enter the brain; this could result in serious side-effects, e.g., seizures and brain swelling, infection and possibly permanent neuropathological changes. Therefore, repeated treatments with these techniques for chronic and diffuse brain diseases affecting multiple brain regions are not practical. Most of these treatments are costly, necessitate hospitalization, and some approaches require anesthesia.

Another approach for circumventing the BBB is direct injection of therapeutic molecules into the cerebrospinal fluid (CSF), the parenchymal space, or other parts of the brain. Several delivery methods have been developed, including: intracerebral (intra-parenchymal), intraventricular, and intrathecal delivery via infusion or convection-enhanced diffusion (CED) pumps. However, any type of direct injection into the brain or intracerebral implant is an invasive and costly procedure, as it requires hospitalization, anesthesia, and often surgery. Moreover, the poor diffusion rates of the therapeutics, particularly large biologics, within brain parenchyma limit the penetration of therapeutics to only small areas surrounding the site of injection/implantation. The correct placement of injections, catheters, and implants is challenging yet crucial to achieve diffusion of the drug to the targeted region of the brain. Additionally, catheters and implants provide a site for infection and/or immune response against the foreign material.

In another attempt to increase delivery across the BBB, CNS drugs have been modified to increase their brain uptake. Such modifications can include a change of their surface charge, a reduction in molecule size, and change to the lipophilicity of the drugs. However, any modifications to increase brain penetration are also likely to alter the overall pharmacology of the drug, including its desired activity and/or specificity. In addition, lipophilic molecules are more prone to being exported from the brain through the P-glycoprotein efflux pump.

Still another approach involves the exploitation of endogenous transport mechanisms across the BBB. Physiological mechanisms that allow transport of large molecules across the BBB can be divided into the highly specific receptor mediated transcytosis (RMT) and the non-specific charge based adsorptive mediated endocytosis pathways. Endocytosis is triggered upon binding of the specific ligand to its receptor, or upon electrostatic interaction between the cationic ligand or drug and the anionic functional groups on the brain endothelial cell surface (luminal side), respectively. Subsequently, the newly formed endosome is transcytosed across the cell to the abluminal side, to release its cargo.

Another alternative is the loosening or disruption of tight junctions. Some modulators of tight junctions include alkylglycerols, bradykinin and several analogues thereof, as well as viruses that modulate expression of proteins involved in maintaining the tight junctions [Erdlenbruch B, 2003; Preston E, 2008; Gan Y. 2013].

Researchers are also continuing to explore osmotic blood-brain barrier opening (OBBBO) [Guillaume D J, 2010; Kroll, 1998; Siegal, 2000; Rapoport, 2001; Ikeda, 2002; Haluska, 2004; Van Vliet, 2007; Marchi, 2007; Lochhead, 2020; Pandit, 2020]. OBBBO involves precisely administering hyperosmotic agents, such as mannitol, to temporarily disrupt the BBB's integrity [Neuwelt, 1979; Burks, 2021; Ikeda, 2002; Chu, 2022; Chu, 2018; Whelan, 2021; Chu, 2020; Lesniak, 2019]. This increases permeability, enabling more efficient drug access to the brain. Osmotic BBBO can be accurately targeted to affected brain regions, minimizing harm to healthy tissue [Chu, 2022; Chu, 2018; Chu, 2020; Lesniak, 2019; Janowski, 2016]. This precision is especially crucial in brain cancer treatment, where sparing healthy neurons while targeting tumor cells is paramount. Compared to some traditional BBB disruption methods, OBBBO is considered safer, reducing the risk of severe side effects and long-term neurological damage [Chu, 2022; Chu, 2018; Chu, 2020; Lesniak, 2019]. OBBBO may also facilitate the delivery of larger therapeutic molecules, including monoclonal antibodies, which are increasingly important in brain cancer treatment. Additionally, combining OBBBO with other therapies like immunotherapy or radiation shows potential for enhancing brain cancer treatment efficacy [Lesniak, 2019; Lesniak, 2019a; Gao, 2021; Zadawski, 2019; Zadawski, 2021].

In summary, overcoming the challenges posed by the blood-brain barrier in brain cancer drug delivery is critical. Osmotic blood-brain barrier opening offers a promising approach to surmounting these obstacles. By enhancing permeability, enabling precise targeting, reducing toxicity, and potentially accommodating larger therapeutic molecules, OBBBO represents a valuable strategy in the fight against brain cancer, inflammatory diseases, genetic diseases, and neurological diseases.

Towards that end, the present disclosure relates to compositions comprising intra-arterially administered hyperosmotic agents that are effective at inducing transient permeabilization of blood-organ barriers, such as the blood-brain barrier (BBB), but also less impermeable but significant barriers between blood and body tumors or tissues and methods of using same to enhance drug delivery to organs, e.g., brain tissue.

### SUMMARY

In one aspect, a method of delivering a substantially improved fraction of at least one therapeutic agent, at least one contrast agent, or both, to an organ of a subject is described, said method comprising:
administering an effective amount of an osmotically active composition to the subject to disrupt a blood-organ barrier at said organ; and
administering a therapeutically effective amount of a formulation comprising the at least one therapeutic agent, at least one contrast agent, or both, to the subject,
   wherein the osmotically active composition induces transient permeabilization of the blood-organ barrier to enhance delivery of the at least one therapeutic agent, at least one contrast agent, or both, to the organ of the subject.

In another aspect, a method of delivering a substantially improved fraction of at least one therapeutic agent, at least one contrast agent, or both, to an organ of a subject is described, said method comprising:
administering an effective amount of an osmotically active composition to the subject to disrupt a blood-organ barrier; and
ceasing the administration of the osmotically active composition to the subject for a time x; and then
immediately administering a therapeutically effective amount of a formulation comprising the at least one therapeutic agent, at least one contrast agent, or both, to the subject,
   wherein the osmotically active composition induces transient permeabilization of the blood-organ barrier to enhance delivery of the at least one therapeutic agent, at least one contrast agent, or both, to the organ of the subject.

In still another aspect, a method of treating cancer, inflammatory or genetic diseases, a neurological disease, or any other disease in a subject is disclosed, said method comprising:
administering an effective amount of an osmotically active composition to the subject to disrupt a blood-organ barrier; and
administering a therapeutically effective amount of a formulation comprising at least one therapeutic agent to the subject,
   wherein the osmotically active composition induces transient permeabilization of the blood-organ barrier to enhance delivery of the at least one therapeutic agent to a an organ of the subject to treat the disease.

In some embodiments, the blood-organ barrier is the blood-brain barrier (BBB) and the organ is the brain of the subject.

Other aspects, features and advantages of the invention will be more fully apparent from the ensuing disclosure and appended claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The accompanying figures are provided by way of illustration and not by way of limitation.

FIG. 1A. Gd enhanced T1 MRI after 25% mannitol-induced BBBO coronal and horizontal view.

FIG. 1B. Gd enhanced T1 MRI after BBBO with 4% saline in 25% mannitol.

FIG. 1C. Box graph showing tissue intensity between brain tissue with intact BBB and BBBO area in OBBBO with 25% mannitol.

FIG. 1D. Box graph showing tissue intensity between brain tissue with intact BBB and BBBO area in OBBBO with 4% saline in 25% Mannitol.

FIG. 1E. Comparisons of ratio of OBBBO area versus BBB intact area between BBBO with 25% mannitol relative to 4% saline in 25% mannitol shows significant differences preferring the second osmotic agent.

FIG. 1F. Comparisons of the area of barrier disruptions of OBBBO area versus BBB intact area between BBBO with 25% mannitol relative to 4% saline in 25% mannitol shows significant differences preferring the second osmotic agent.

FIG. 2A. Turbo RARE images 3 and 7 days after BBBO showing no indication of brain damage.

FIG. 2B. No Gd enhancement on T1-weighted images was observed in the brain, suggesting that the BBB was resealed.

FIG. 3A. Gadolinium-enhanced T1 MRI coronal view, after IA OBBBO using 25% mannitol+4% NaCl.

FIG. 3B. Gadolinium-enhanced T1 MRI coronal view, 3 days after IA OBBBO using 25% mannitol+4% NaCl.

FIG. 3C. Gadolinium-enhanced T1 MRI coronal view, 14 days after IA OBBBO using 25% mannitol+4% NaCl.

FIG. 3D. PET/CT coronal slice images of accumulation of ⁸⁹Zr-bevacizumab in a C57BL6 mouse after IA OBBBO using 4% saline in 25% mannitol and IA injection of radiolabeled bevacizumab.

FIG. 3E. PET/CT horizontal slice images of accumulation of ⁸⁹Zr-bevacizumab in a C57BL6 mouse after IA OBBBO using 4% saline in 25% mannitol and IA injection of radiolabeled bevacizumab.

FIG. 3F. Statistic graphics of standardized uptake of ⁸⁹Zr-bevacizumab in brain hemispheres.

FIG. 3G. Statistical graphics of biodistribution of ⁸⁹Zr-bevacizumab in brain hemispheres.

FIG. 4A. Mass spectrometry results showing a control sample without DFO, having one peak corresponding with three glycoforms of bevacizumab.

FIG. 4B. Mass spectrometry results showing a sample with attached DFO, wherein the first peak represents bevacizumab without DFO, the second peak is bevacizumab with one DFO molecule, the third peak is bevacizumab with two DFO molecules, the fourth peak is bevacizumab with three DFO molecules, etc.

FIG. 5A. Right after BBBO animals in both groups were injected with radiolabeled Bevacizumab intraarterially and transferred into PET-CT for 30 min scan. In the 25% mannitol group, only a small amount of Gd within the brain parenchyma was observed. The upper two rows are coronal, and the lower two rows are horizontal projections.

FIG. 5B. In the group receiving 4% saline in 25% mannitol, the area and intensity signifying the amount of Gd in the brain parenchyma is much higher.

FIG. 6A. SUVmax (maximum standardized uptake value) in the BBBO with 25% mannitol group.

FIG. 6B. SUVmax (maximum standardized uptake value) in the BBBO with 4% saline in 25% mannitol group.

FIG. 6C. The ipsilateral vs contralateral hemisphere ratio of the 25% mannitol group relative to the 4% saline in 25% mannitol group.

FIG. 6D. Percentage of the total injection dose (ID %) per gram tissue weight in the BBBO with 25% mannitol group.

FIG. 6E. Percentage of the total injection dose (ID %) per gram tissue weight in the BBBO with 4% saline in 25% mannitol group.

FIG. 6F. The ipsilateral vs contralateral hemisphere ratio of the 25% mannitol group relative to the 4% saline in 25% mannitol group.

FIG. 7A. Intra-arterial delivery of Luc mRNA and antibodies to head and neck. Schematic diagram of targeted delivery via external carotid artery (ECA) route.

FIG. 7B. Representative BLI image of mice received IV and IA administration of Luc mRNA at different timepoints post injection.

FIG. 7C. Quantification of BLI signal showed the Luc expression over time.

FIG. 7D. PET/CT images of radiolabeled antibodies (Zr 89-bevacizumab) via different delivery routes.

FIG. 7E. Ex biodistribution of Zr 89 radioactivity in the organs.

FIG. 8A. Coronal PET/CT of the head in a mouse after OBBBO with various doses and routes of delivery Bevacizumab.

FIG. 8B. Biodistribution of Bevacizumab in hemispheres by route of administration (ROA) and dose. (*P<0.05, ** P<0.01, *** P<0.001, **** P<0.0001)

FIG. 8C. Biodistribution of Bevacizumab in hemispheres by ROA, dose, and buffer. (*P<0.05, ** P<0.01, *** P<0.001, **** P<0.0001)

### DETAILED DESCRIPTION, AND PREFERRED EMBODIMENTS THEREOF

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents, and other references mentioned herein are incorporated by reference in their entirety. The materials, methods, and examples disclosed herein are illustrative only and not intended to be limiting.

“About” and “approximately” are used to provide flexibility to a numerical range endpoint by providing that a given value may be “slightly above” or “slightly below” the endpoint without affecting the desired result, for example, +/−5%.

The phrase “in one embodiment” or “in some embodiments” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising.” “consisting of” and “consisting essentially of.” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Subject” and “patient” as used herein interchangeably refers to any vertebrate, including, but not limited to, an animal (e.g., a bear, cow, cattle, pig, camel, llama, horse, goat, rabbit, sheep, hamster, guinea pig, cat, tiger, lion, cheetah, jaguar, bobcat, mountain lion, dog, wolf, coyote, rat, mouse, and a non-human primate (for example, a monkey, such as a cynomolgus or rhesus monkey, chimpanzee, etc.) and a human. In some embodiments, the subject is a human.

As defined herein, “neurological diseases” can be categorized according to the primary location affected, the primary type of dysfunction involved, or the primary type of cause. The broadest division is between central nervous system (CNS) disorders and peripheral nervous system (PNS) disorders. The Merck Manual lists brain, spinal cord and nerve disorders in the following overlapping categories, all of which are contemplated by the invention: brain damage according to cerebral lobe, i.e., Frontal lobe damage, Parietal lobe damage, Temporal lobe damage, and Occipital lobe damage; brain dysfunction according to type: Aphasia (language), Dysarthria (speech), Apraxia (patterns or sequences of movements), Agnosia (identifying things/people), and Amnesia (memory); spinal cord disorders; peripheral neuropathy & other peripheral nervous system disorders; cranial nerve disorders such as Trigeminal neuralgia; autonomic nervous system disorders, such as dysautonomia and Multiple System Atrophy; seizure disorders, such as epilepsy; movement disorders of the central & peripheral nervous system, such as Parkinson's disease, essential tremor, amyotrophic lateral sclerosis (ALS), Tourette's Syndrome, multiple sclerosis & various types of peripheral neuropathy; sleep disorders, such as narcolepsy; migraines and other types of headache, such as cluster headache and tension headache; lower back and neck pain; central Neuropathy (see Neuropathic pain); and neuropsychiatric illnesses (diseases and/or disorders with psychiatric features associated with known nervous system injury, underdevelopment, biochemical, anatomical, or electrical malfunction, and/or disease pathology e.g., Attention deficit hyperactivity disorder, Autism, Tourette's Syndrome & some cases of Obsessive compulsive disorder as well as the neurobehavioral associated symptoms of degeneratives of the nervous system such as Parkinson's disease, Essential tremor, Huntington's disease, Alzheimer's disease, Multiple sclerosis & organic psychosis).

As used herein. “therapeutically effective amount” means the amount of a compound that, when administered to a patient for treating a disease, is sufficient to effect such treatment for the disease, e.g., the amount of such a substance that produces some desired local or systemic effect at a reasonable benefit/risk ratio applicable to any treatment, e.g., is sufficient to ameliorate at least one sign or symptom of the disease, e.g., to prevent progression of the disease or condition, e.g., prevent tumor growth, decrease tumor size, induce tumor cell apoptosis, reduce tumor angiogenesis, prevent metastasis. When administered for preventing a disease, the amount is sufficient to avoid or delay onset of the disease. The “therapeutically effective amount” will vary depending on the compound, its therapeutic index, solubility, the disease and its severity and the age, weight, etc., of the patient to be treated, and the like. Administration of a therapeutically effective amount of a compound may require the administration of more than one dose of the compound.

The term “antibody” as used herein refers to immunoglobulin molecules and immunologically active portions of immunoglobulin (Ig) molecules, i.e., molecules that contain an antigen-binding site that specifically binds (immunoreacts with) an antigen, comprising at least one, and preferably two, heavy (H) chain variable regions (abbreviated herein as VH), and at least one and preferably two light (L) chain variable regions (abbreviated herein as VL). Such antibodies include, but are not limited to, polyclonal, monoclonal, chimeric, single chain, Fab, Fab′ and F(ab′) 2 fragments, and a Fab expression library. The VH and VL regions can be further subdivided into regions of hypervariability, termed “complementarity determining regions” (“CDR”), interspersed with regions that are more conserved, termed “framework regions” (FR). The extent of the framework region and CDR's has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917, which are incorporated herein by reference). Each VH and VL is composed of three CDR's and four FRs, arranged from amino-terminus to carboxy-terminus in the following order: FR1, CDR1, FR2, CDR2, FR3, CDR3, FR4. In general, antibody molecules obtained from humans relates to any of the classes IgG. IgM, IgA, IgE and IgD, which differ from one another by the nature of the heavy chain present in the molecule. Certain classes have subclasses as well, such as IgG1, IgG2, and others. Furthermore, in humans, the light chain may be a kappa chain or a lambda chain. Reference herein to antibodies includes a reference to all such classes, subclasses and types of human antibody species. Therapeutically useful antibodies further include “humanized” monoclonal antibodies, fully human antibodies, bispecific antibodies, and heteroconjugate antibodies.

As used herein, an “organ” includes, but is not limited to, brain, heart, stomach, pancreas, knee, liver, kidney, lung, thymus, adrenals, skin, bladder, reproductive organs, salivary glands, tongue, jaw muscles, intestine, colon, spleen, brain, the like or parts thereof.

As used herein, a “blood-organ barrier” is intended to correspond to any blood-to-organ tissue transition of the subject including, but is not limited to, the blood brain barrier (BBB), blood pancreas barrier, blood tumor barrier, blood muscle barrier, and others. As used herein, the “organ” includes, but is not limited to, skin, salivary glands, tongue, brain, pancreas, and jaw muscles, and any other organ of the body. It should be appreciated by the person skilled in the art that a substantially improved fraction of the therapeutic agent that is delivered is intended to cross the blood-organ barrier into the specific organ. Further, it should be appreciated that the therapeutic agent may be delivered preferentially to only a portion of the organ, for example, ipsilateral brain tissue over contralateral brain tissue.

As used herein, a “substantially improved fraction” corresponds to an improvement of the delivery of the at least one therapeutic agent across the blood-organ barrier, relative to any prior art methods, of at least about 2-fold, 3-fold, 4-fold, 5-fold, 6-fold, 7-fold, 8-fold, 9-fold, 10-fold, 20-fold, 30-fold, 40-fold, 50-fold, 100-fold, 200-fold or more.

As used herein, to “disrupt” is to at least partially open a blood-organ barrier.

As used herein, “contrast agents” are a group of contrast media used to improve the visibility of internal body structures in, but not limited to, magnetic resonance imaging (MRI). The most commonly used compounds for contrast enhancement are gadolinium-based. MRI contrast agents alter the relaxation times of atoms within body tissues where they are present after oral or intravenous administration. In MRI scanners, sections of the body are exposed to a very strong magnetic field, aligning the magnetic moments of certain nuclei, primarily hydrogen. A radiofrequency pulse is then applied, tipping these aligned nuclei out of equilibrium. After the pulse stops, the nuclei relax, emitting signals that are detected by the scanner and are mathematically converted into an image. The MRI image can be weighted in different ways to enhance or suppress specific signals, enabling clear differentiation between various tissues types.

As used herein, “enhanced delivery” of the at least one therapeutic agent, at least one contrast agent, or both, to the organ corresponds to the delivery of a substantially improved fraction of the at least one therapeutic agent, at least one contrast agent, or both, contained in the formulation to the organ of intended delivery, relative to delivery methods known in the prior art. For example, if using a method of the prior art ensured that a maximum of 0.1 wt % of an active agent can cross the blood-organ barrier, the methods described herein can increase that amount of the active agent that crosses the blood-organ barrier, e.g., a BBB, at least about 2-fold, 3-fold, 4-fold, 5-fold, 6-fold, 7-fold, 8-fold, 9-fold, 10-fold, 20-fold, 30-fold, 40-fold, 50-fold, 100-fold, 200-fold, or more, relative to said method of the prior art.

Broadly, the present disclosure relates to methods of administering at least one therapeutic agent, at least one contrast agent, or both, to the brain or central nervous system of a subject to treat diseases such as cancer, e.g., brain cancer or head and neck cancer, an inflammatory disease, a neurological disease, a mental disease, genetic disease, muscle disease or other organ disease. The therapeutic agent may be for example any agent suitable for administration to the brain or any other organ including a chemotherapeutic agent, gene therapy agent or a neurotherapeutic agent. Chemotherapeutic agents include any agents known to be therapeutic against cancers including brain cancers and head and neck cancers or cancers that have metastasized to the brain. Gene therapy agents include, but are not limited to, viral or non-viral agents such as AAV, lentivirus or nanoparticle-based viral free agents. Neurotherapeutic agents include, for example, PDGF, VEGF, dopamine and any agent known to be therapeutic to neurological diseases such as Alzheimer's disease, Parkinson's disease, stroke, and the like. It should be appreciated by the person skilled in the art however that the method described herein can be used to deliver any desired therapeutic agent, any desired contrast agent, or both, to any organ of a subject.

Advantageously, using the methods described herein, a high osmotic effect can be obtained by a combination of agents that are safe for the brain and elsewhere in the body. Accordingly, an osmotic process can universally improve the delivery of therapeutic agents, contrast agents, or both, to any organs or tissues in a subject.

In a first aspect, a method of delivering a substantially improved fraction of at least one therapeutic agent, at least one contrast agent, or both, to an organ of a subject is described, said method comprising:
administering an effective amount of an osmotically active composition to the subject to disrupt a blood-organ barrier at said organ; and
administering a therapeutically effective amount of a formulation comprising the at least one therapeutic agent, at least one contrast agent, or both, to the subject,
   wherein the osmotically active composition induces transient permeabilization of the blood-organ barrier to enhance delivery of the at least one therapeutic agent, at least one contrast agent, or both, to the organ of the subject.

In some embodiments of the first aspect, a method of delivering a substantially improved fraction of at least one therapeutic agent to an organ of a subject is described, said method comprising:
administering an effective amount of an osmotically active composition to the subject to disrupt a blood-organ barrier at said organ; and
administering a therapeutically effective amount of a formulation comprising the at least one therapeutic agent to the subject,
   wherein the osmotically active composition induces transient permeabilization of the blood-organ barrier to enhance delivery of the at least one therapeutic agent to the organ of the subject.

In some embodiments of the first aspect, a method of delivering a substantially improved fraction of at least one contrast agent to an organ of a subject is described, said method comprising:
administering an effective amount of an osmotically active composition to the subject to disrupt a blood-organ barrier at said organ; and
administering a therapeutically effective amount of a formulation comprising the at least one contrast agent to the subject,
   wherein the osmotically active composition induces transient permeabilization of the blood-organ barrier to enhance delivery of the at least one contrast agent to the organ of the subject.

In some embodiments, the blood-organ barrier is the BBB and the organ is the brain. In some embodiments, the blood-organ barrier, e.g., the BBB, is disrupted at a specific, local arterial region/territory by catheter-based administration of the osmotically active composition. In some embodiments, the osmotically active composition is administered intraarterially via a catheter placed in an artery that is directly feeding the organ or a portion of same. In some embodiments, the osmotically active composition is administered intraarterially. In some embodiments, the osmotically active composition is administered intraarterially via the common carotid artery (CCA). In some embodiments, a contrast agent may be administered in combination with the osmotically active composition or sequentially to enable visualization of the location and formation of the disrupting of the blood-organ barrier, e.g., the BBB. In some embodiments where the contrast agent is used, once the blood-organ disruption has been detected, the formulation comprising the at least one therapeutic agent, at least one contrast agent, or both, may be administered by intraarterial infusion, e.g., through the same or separate catheter, at the same site or proximal the site of disruption or the formulation can be administered systemically (e.g., intravenously, intraperitoneally, etc.). In some embodiments, the formulation comprising the at least one therapeutic agent, at least one contrast agent, or both, may be administered by intraarterial infusion, e.g., through the same or separate catheter, at the same site or proximal the site of disruption or the formulation can be administered systemically (e.g., intravenously, intraperitoneally, etc.). In some embodiments, the formulation is administered intraarterially via a catheter placed in an artery that is in directly feeding the organ or a portion thereof. In some embodiments, the at least one therapeutic agent is intended to pass through the subject's blood-organ barrier, e.g., BBB, to treat a disease such as cancer, e.g., brain cancer or head and neck cancer, an inflammatory disease, a genetic disease, a neurological disease, a mental disease or any other disease of an organ. In some embodiments, the cancer is brain cancer or cancers that have metastasized to the brain. In some embodiments, the cancer is head and neck cancer. In some embodiments, the cancer is body cancer.

Although the method of the first aspect is disclosed as administering the osmotically active composition to disrupt the blood-organ barrier, e.g., the BBB, followed by administration of the formulation comprising at least one therapeutic agent, at least one contrast agent, or both, it should be appreciated by the person skilled in the art that the osmotically active composition can be administered at the same time as, or with, the formulation comprising at least one therapeutic agent, at least one contrast agent, or both.

In some other embodiments, the administration of the osmotically active composition to the subject to disrupt the blood-organ barrier, e.g., BBB, is ceased for a time x before a therapeutically effective amount of a formulation comprising at least one therapeutic agent, at least one contrast agent, or both, is administered to the subject. The cessation of the administration of the osmotically active composition for a time x before administration of the formulation is hereinafter referred to as “pretreatment.”

In some embodiments of the first aspect, a method of delivering a substantially improved fraction of at least one therapeutic agent, at least one contrast agent, or both, to an organ of a subject is described, said method comprising:
administering an effective amount of an osmotically active composition to the subject to disrupt a blood-organ barrier; and
ceasing the administration of the osmotically active composition to the subject for a time x; and then
immediately administering a therapeutically effective amount of a formulation comprising at least one therapeutic agent, at least one contrast agent, or both, to the subject,
   wherein the osmotically active composition induces transient permeabilization of the blood-organ barrier to enhance delivery of the at least one therapeutic agent, at least one contrast agent, or both, to the organ of the subject.

In some embodiments of the first aspect, a method of delivering a substantially improved fraction of at least one therapeutic agent to an organ of a subject is described, said method comprising:
administering an effective amount of an osmotically active composition to the subject to disrupt a blood-organ barrier; and
ceasing the administration of the osmotically active composition to the subject for a time x; and then
immediately administering a therapeutically effective amount of a formulation comprising at least one therapeutic agent to the subject,
   wherein the osmotically active composition induces transient permeabilization of the blood-organ barrier to enhance delivery of the at least one therapeutic agent to the organ of the subject.

In some embodiments of the first aspect, a method of delivering a substantially improved fraction of at least one contrast agent to an organ of a subject is described, said method comprising:
administering an effective amount of an osmotically active composition to the subject to disrupt a blood-organ barrier; and
ceasing the administration of the osmotically active composition to the subject for a time x; and then
immediately administering a therapeutically effective amount of a formulation comprising at least one contrast agent to the subject,
   wherein the osmotically active composition induces transient permeabilization of the blood-organ barrier to enhance delivery of the at least one contrast agent to the organ of the subject.

In some embodiments, the blood-organ barrier is the BBB and the organ is a brain. In some embodiments, the osmotically active composition is administered intraarterially. In some embodiments, the osmotically active composition is administered intraarterially via a catheter placed in an artery that is directly feeding the organ or a portion thereof. In some embodiments, the formulation comprising at least one therapeutic agent, at least one contrast agent, or both, may be administered by intraarterial infusion. In some embodiments, the formulation is administered intraarterially via a catheter placed in an artery that is directly feeding the organ or a portion thereof. In some embodiments, the formulation is administered, e.g., intraarterially, such that the formulation substantially displaces the blood in the artery and downstream capillary vessels. In some embodiments, the time x between the stopping of the administration of the osmotically active composition to the subject and the administration of the formulation comprising at least one therapeutic agent, at least one contrast agent, or both, to the subject is in a range from about 1 sec to about 120 min, or about 1 sec to 60 min, or about 1 sec to about 30 min, or about 1 sec to about 10 min, or about 1 sec to 5 min, or about 1 sec to about 3 min, or about 1 sec to about 1 min, or about 1 sec to about 30 sec. In some embodiments, the same or a different catheter is used to administer the osmotically active composition and the formulation comprising the at least one therapeutic agent, at least one contrast agent, or both. In some embodiments, a balloon catheter is used to position the balloon within an artery proximal to the target site, i.e., the organ. The balloon is inflated immediately before and during the infusion of the therapeutic agent, at least one contrast agent, or both, to increase the dwelling time, thereby enhancing absorption of at least one therapeutic agent, at least one contrast agent, or both. In some embodiments, the at least one therapeutic agent is intended to pass through the subject's blood-organ barrier, e.g., BBB, to treat a disease such as the cancer, e.g., brain cancer or head and neck cancer, an inflammatory disease, a genetic disease, a neurological disease, a mental disease or any other disease associated with an organ. In some embodiments, the cancer is brain cancer or cancers that have metastasized to the brain. In some embodiments, the cancer is body cancer.

Osmotic opening using 25% mannitol has been known for several decades, and it is considered clinically safe, but disadvantageously has a high variability and insufficient efficacy so it has never become a routine clinical treatment. Advantageously, the method of the first aspect increases osmotic power, is safe, and boosts efficacy of every blood-organ barrier, e.g., including the BBB, in the subject.

Without being bound by theory, the delivery of the at least one therapeutic agent, at least one contrast agent, or both, comprised in the formulation, via an intraarterial catheter directly feeding the organ, following pretreatment of the blood-organ barrier, is experimentally more effective relative to other delivery means (e.g., IV delivery). This increased efficacy is attributed to the substantial displacement of the blood during the IA delivery, ensuring that a substantially improved fraction of the at least one therapeutic agent, at least one contrast agent, or both, is delivered to the organ directly, relative to methods known in the prior art, wherein the displacement minimizes contact of the formulation comprising at least one therapeutic agent, at least one contrast agent, or both, with blood components, thereby avoiding any detrimental effects of such interactions on drug extravasation. In some embodiments, it is not possible to take advantage of the displacement advantage if the formulation comprising the at least one therapeutic agent, at least one contrast agent, or both, is subsequently administered intravenously, instead of the intraarterial administration discussed herein.

In some embodiments, using the method of the first aspect and the osmotically active composition described herein, the uptake of the therapeutic agent in the ipsilateral hemisphere of an organ is statistically greater than the uptake of the therapeutic agent in the contralateral hemisphere of the organ. In some embodiments, using the method of the first aspect and the osmotically active composition described herein, the uptake of the therapeutic agent in the ipsilateral hemisphere of an organ is at least two, three, or four times greater than the uptake of the therapeutic agent in the contralateral hemisphere of the organ. This is an advantage over prior art methods using an osmotically active composition consisting of 25% mannitol in water, which often have no significant statistical difference between the uptake at the two hemispheres.

In some embodiments, the method of the first aspect can also be used for liquid biopsy, so at the time of the procedure blood can be drawn for molecular markers of diseases.

### Osmotically Active Composition

In some embodiments, the osmotically active composition comprises, consists of, or consists essentially of at least one osmotically active species and water. In some embodiments, the at least one osmotically active species is selected from mannitol, dextrose, propylene glycol, glycerol, sorbitol, trehalose, erythritol, CaCl₂), KBr, KCl, LiCl, NaCl, NaBr, Na₂SO₄, or any combination thereof. In some embodiments, the osmotically active composition comprises, consists of, or consists essentially of at least one osmotically active species, at least one additional component, and water.

In some other embodiments, the osmotically active composition comprises, consists of, or consists essentially of at least one osmotically active agent, at least one ionic osmotically agent, and water. In some embodiments, the osmotically active composition comprises, consists of, or consists essentially of at least one osmotically active agent, at least one ionic osmotically active agent, at least one additional component, and water.

The at least one osmotically active agent includes, but is not limited to, mannitol, dextrose, propylene glycol, glycerol, mannitol, sorbitol, trehalose, erythritol, and any combination thereof. In some embodiments, the at least one osmotically active agent comprises, consists of, or consists essentially of mannitol. Mannitol is non-metabolizable and well-tolerated by the body and, because of its size, has strong osmotic effect. In some embodiments, the at least one osmotically active agent comprises, consists of, or consists essentially of D-mannitol. In some embodiments, the osmotically active composition comprises, consists of, or consists essentially of about 15% (w/w) to about 35% (w/w) of the at least one osmotically active agent. In some embodiments, the osmotically active composition comprises, consists of, or consists essentially of about 20% (w/w) to about 30% (w/w) of the at least one osmotically active agent. In some embodiments, the osmotically active composition comprises, consists of, or consists essentially of about 21% (w/w) to about 29% (w/w) of the at least one osmotically active agent. In some embodiments, the osmotically active composition comprises, consists of, or consists essentially of about 22% (w/w) to about 28% (w/w) of the at least one osmotically active agent. In some embodiments, the osmotically active composition comprises, consists of, or consists essentially of about 23% (w/w) to about 27% (w/w) of the at least one osmotically active agent. In some embodiments, the osmotically active composition comprises, consists of, or consists essentially of about 24% (w/w) to about 26% (w/w) of the at least one osmotically active agent. In some embodiments, the osmotically active composition comprises, consists of, or consists essentially of about 24.5% (w/w) to about 25.5% (w/w) of the at least one osmotically active agent. In some embodiments, the osmotically active composition comprises, consists of, or consists essentially of about 25% (w/w) of the at least one osmotically active agent. In some embodiments, the osmotically active composition comprises, consists of, or consists essentially of about 15% (w/w) to about 35% (w/w) mannitol. In some embodiments, the osmotically active composition comprises, consists of, or consists essentially of about 20% (w/w) to about 30% (w/w) mannitol. In some embodiments, the osmotically active composition comprises, consists of, or consists essentially of about 21% (w/w) to about 29% (w/w) mannitol. In some embodiments, the osmotically active composition comprises, consists of, or consists essentially of about 22% (w/w) to about 28% (w/w) mannitol. In some embodiments, the osmotically active composition comprises, consists of, or consists essentially of about 23% (w/w) to about 27% (w/w) mannitol. In some embodiments, the osmotically active composition comprises, consists of, or consists essentially of about 24% (w/w) to about 26% (w/w) mannitol. In some embodiments, the osmotically active composition comprises, consists of, or consists essentially of about 24.5% (w/w) to about 25.5% (w/w) mannitol. In some embodiments, the osmotically active composition comprises, consists of, or consists essentially of about 25% (w/w) mannitol.

In some embodiments, the at least one ionic osmotically active agent includes, but is not limited to, MgCl₂, MgBr₂, CaCl₂, CaBr₂, KBr, KCl, LiCl, LiBr, NaCl, NaBr, Na₂SO₄, K₂SO₄, and any combination thereof. In some embodiments, the at least one ionic osmotically active agent comprises, consists of, or consists essentially of NaCl. In some embodiments, the osmotically active composition comprises, consists of, or consists essentially of about 2% (w/w) to about 24% (w/w), or more, of the at least one ionic osmotically active agent. In some embodiments, the osmotically active composition comprises, consists of, or consists essentially of about 2% (w/w) to about 20% (w/w) of the at least one ionic osmotically active agent. In some embodiments, the osmotically active composition comprises, consists of, or consists essentially of about 2% (w/w) to about 15% (w/w) of the at least one ionic osmotically active agent. In some embodiments, the osmotically active composition comprises, consists of, or consists essentially of about 2% (w/w) to about 10% (w/w) of the at least one ionic osmotically active agent. In some embodiments, the osmotically active composition comprises, consists of, or consists essentially of about 3% (w/w) to about 5% (w/w) of the at least one ionic osmotically active agent. In some embodiments, the osmotically active composition comprises, consists of, or consists essentially of about 3.5% (w/w) to about 4.5% (w/w) of the at least one ionic osmotically active agent. In some embodiments, the osmotically active composition comprises, consists of, or consists essentially of about 4% (w/w) of the at least one ionic osmotically active agent. In some embodiments, the osmotically active composition comprises, consists of, or consists essentially of about 2% (w/w) to about 6% (w/w) NaCl. In some embodiments, the osmotically active composition comprises, consists of, or consists essentially of about 3% (w/w) to about 5% (w/w) NaCl. In some embodiments, the osmotically active composition comprises, consists of, or consists essentially of about 3.5% (w/w) to about 4.5% (w/w) NaCl. In some embodiments, the osmotically active composition comprises, consists of, or consists essentially of about 4% (w/w) NaCl.

In some embodiments, the osmotically active composition can comprise, consist of, or consist essentially of mannitol, NaCl, and water. In some embodiments, the osmotically active composition can comprise, consist of, or consist essentially of about 15% (w/w) to about 35% (w/w) mannitol, about 2% (w/w) to about 6% (w/w) NaCl, and water. In some embodiments, the osmotically active composition can comprise, consist of, or consist essentially of about 20% (w/w) to about 30% (w/w) mannitol, about 3% (w/w) to about 5% (w/w) NaCl, and water. In some embodiments, the osmotically active composition can comprise, consist of, or consist essentially of about 23% (w/w) to about 27% (w/w) mannitol, about 3.5% (w/w) to about 4.5% (w/w) NaCl, and water. In some embodiments, the osmotically active composition can comprise, consist of, or consist essentially of about 24% (w/w) to about 26% (w/w) mannitol, about 3.5% (w/w) to about 4.5% (w/w) NaCl, and water. In some embodiments, the osmotically active composition can comprise, consist of, or consist essentially of about 24.5% (w/w) to about 25.5% (w/w) mannitol, about 3.5% (w/w) to about 4.5% (w/w) NaCl, and water. In some embodiments, the osmotically active composition can comprise, consist of, or consist essentially of about 25% (w/w) mannitol, about 4% (w/w) NaCl, and water.

In some embodiments, the blood-organ barrier is the BBB and the osmolality of the osmotically active composition is greater than or equal to about 1,372 mOsm/L, or greater than about 1,400 mOsm/L. or greater than about 1,500 mOsm/L, or greater than about 1,600 mOsm/L, or greater than about 1,700 mOsm/L, or greater than about 1,800 mOsm/L, or greater than about 1,900 mOsm/L, or greater than about 2.000 mOsm/L, or greater than about 2,100 mOsm/L, or greater than about 2,200 mOsm/L, or greater than about 2,300 mOsm/L, or greater than about 2,400 mOsm/L, or greater than about 2,500 mOsm/L, or greater than about 2,600 mOsm/L, or greater than about 2,700 mOsm/L, or greater than about 2,742 mOsm/L. In some embodiments, an osmotically active composition comprising 25% mannitol and 4% NaCl has an osmolality of 2,742 mOsm/L. In some embodiments, the blood-organ barrier is the BBB and the osmotic power (pressure) or osmolality of the at least one osmotically active agent is in a range of about 1,000 to about 1,600 mOsm/L and the osmolality of the at least one ionic osmotically active agent is in a range of about 1,000 to about 1,600 mOsm/L. In some embodiments, the blood-organ barrier is the BBB and the osmotic power (pressure) or osmolality of the at least one osmotically active agent is in a range of about 1,000 to about 1,600 mOsm/L and the osmolality of the at least one ionic osmotically active agent is in a range of about 1,000 to about 1,600 mOsm/L, so long as the total osmolality of the osmotically active composition is greater than or equal to about 2,742 mOsm/L. In some embodiments, the blood-organ barrier is the BBB and the osmotic power (pressure) or osmolality of the at least one osmotically active agent is in a range of about 1,100 to about 1,500 mOsm/L and the osmolality of the at least one ionic osmotically active agent is in a range of about 1,100 to about 1,500 mOsm/L. In some embodiments, the blood-organ barrier is the BBB and the osmotic power (pressure) or osmolality of the at least one osmotically active agent is in a range of about 1,100 to about 1.500 mOsm/L and the osmolality of the at least one ionic osmotically active agent is in a range of about 1,100 to about 1,500 mOsm/L, so long as the total osmolality of the osmotically active composition is greater than or equal to about 2,742 mOsm/L. In some embodiments, the blood-organ barrier is the BBB and the osmotic power (pressure) or osmolality of the at least one osmotically active agent is in a range of about 1,200 to about 1,400 mOsm/L and the osmolality of the at least one ionic osmotically active agent is in a range of about 1,200 to about 1,400 mOsm/L. In some embodiments, the blood-organ barrier is the BBB and the osmotic power (pressure) or osmolality of the at least one osmotically active agent is in a range of about 1,200 to about 1,400 mOsm/L and the osmolality of the at least one ionic osmotically active agent is in a range of about 1,200 to about 1,400 mOsm/L, so long as the total osmolality of the osmotically active composition is greater than or equal to about 2,742 mOsm/L. In some embodiments, the blood-organ barrier is the BBB and the osmotic power (pressure) or osmolality of the at least one osmotically active agent is greater than or equal to about 1,372 mOsm/L. In some embodiments, the blood-organ barrier is the BBB and the osmolality of the at least one ionic osmotically active agent is greater than or equal to about 1.370 mOsm/L. In some embodiments, the blood-organ barrier is the BBB and the osmotic power (pressure) or osmolality of the at least one osmotically active agent is greater than or equal to about 1.372 mOsm/L and the osmolality of the at least one ionic osmotically active agent is greater than or equal to about 1,370 mOsm/L.

In some embodiments, the blood-organ barrier is not the BBB and the threshold for breaking through the barrier is lower than that for the BBB. In some embodiments, the blood-organ barrier is not the BBB and the osmolality of the osmotically active composition is greater than or equal to about 312 mOsm/L. In some embodiments, the blood-organ barrier is not the BBB and the osmotic power (pressure) or osmolality of the at least one osmotically active agent is in a range of about 100 to about 200 mOsm/L and the osmolality of the at least one ionic osmotically active agent is in a range of about 100 to about 200 mOsm/L, so long as the total osmolality of the osmotically active composition is greater than or equal to about 312 mOsm/L. In some embodiments, the blood-organ barrier is not the BBB and the osmotic power (pressure) or osmolality of the at least one osmotically active agent is in a range of about 50 to about 150 mOsm/L and the osmolality of the at least one ionic osmotically active agent is in a range of about 150 to about 250 mOsm/L, so long as the total osmolality of the osmotically active composition is greater than or equal to about 312 mOsm/L. In some embodiments, the blood-organ barrier is not the BBB and the osmotic power (pressure) or osmolality of the at least one osmotically active agent is in a range of about 150 to about 250 mOsm/L and the osmolality of the at least one ionic osmotically active agent is in a range of about 50 to about 150 mOsm/L, so long as the total osmolality of the osmotically active composition is greater than or equal to about 312 mOsm/L.

In some embodiments, the osmotically active composition further comprises at least one additional component including, but not limited to, any osmotically active substance, a physiologically acceptable buffering agent, an antimicrobial agent, a stabilizer, a preservative, an imaging agent, and/or a contrast agent, as understood by the person skilled in the art. Osmotically active substances, in addition to the at least one osmotically active agent and the at least one ionic osmotically active agent described herein, include all water-soluble substances acceptable for use in pharmaceutics, such as the water-soluble excipients mentioned in pharmacopoeias, in “Hager” and “Remington Pharmaceutical Science” or in other literature (Sareen. R., Jain, N., Kumar, D., Current Drug Delivery, 9, (2012), 285-296). It is possible in particular to use water-soluble salts of inorganic or organic acids or nonionic organic substances with high solubility in water, such as carbohydrates, especially sugars, sugar alcohols or amino acids. For example, the osmotically active substances may be selected from inorganic salts such as carbonates, bicarbonates, phosphates, hydrogen phosphates or dihydrogen phosphates, acetates, succinates, benzoates, citrates or ascorbates of alkali metals or alkaline earth metals such as lithium, sodium, potassium, magnesium and calcium. It is further possible to use pentoses such as arabinose, ribose or xylose, hexoses such as glucose, fructose, galactose or mannose, disaccharides such as sucrose, maltose or lactose or trisaccharides such as raffinose. Water-soluble amino acids include glycine, leucine, alanine or methionine.

### Formulations Comprising the at Least One Therapeutic Agent, at Least One Contrast Agent, or Both

In some embodiments, the formulation comprises at least one therapeutic agent. In some embodiments, the formulation comprises at least one contrast agent, as described herein. In some other embodiments, the formulation comprises at least one therapeutic agent and at least one contrast agent.

In some embodiments, the formulation comprising at least one therapeutic agent, at least one contrast agent, or both, includes a sterile aqueous or non-aqueous solution, suspension, or emulsion, and in particular, formulations suitable for intra-arterial infusion or injection via a catheter. In some embodiments, the formulation comprises an aqueous carrier including, but not limited to, water, alcoholic/aqueous solutions, emulsions or suspensions, including saline and buffered media. In some embodiments, the formulation comprises a non-aqueous carrier including, but not limited to, propylene glycol, glycerol, polyethylene glycol, castor oil, DMSO, vegetable oils such as olive oil, and injectable organic esters such as ethyl oleate.

In some embodiments, the formulation further comprises at least one pharmaceutically acceptable excipient including, but not limited to, preservatives, antimicrobial or antibacterial agents (e.g., sodium hypochlorite, iodine potassium iodide, ethyl alcohol, chlorhexidine, parabens, chlorobutanol, phenol, ascorbic acid, thimerosal), anti-oxidants (e.g., ascorbic acid or sodium bisulfite), chelating agents (e.g., ethylenediaminetetraacetic acid (EDTA)), electrolytes, diluents (e.g., dextrose solution, Finger's solution, or human serum albumin), buffering agents (e.g., bicarbonates, acetates, citrates or phosphates), surfactants, and inert gases and the like. It is understood by the person skilled in the art that if the at least one therapeutic agent is insoluble or partially insoluble in the carrier, e.g., aqueous or non-aqueous carrier, then the at least one pharmaceutically acceptable excipient further includes a component that solubilizes or encapsulates the at least one therapeutic agent therein.

The formulations must be sterile and should be fluid to the extent that easy syringeability exists. It must be stable under the conditions of manufacture and storage and must be preserved against the contaminating action of microorganisms such as bacteria and fungi. The proper fluidity can be maintained, for example, by the use of a coating such as lecithin, by the maintenance of the required particle size in the case of dispersion and by the use of surfactants.

Sterile injectable solutions can be prepared by incorporating the at least one therapeutic agent in the required amount in an appropriate solvent, optionally with one or a combination of pharmaceutically acceptable excipient enumerated above, followed by filtered sterilization.

It should also be appreciated that in some embodiments, the formulation can comprise (i) at least one therapeutic agent, at least one contrast agent, or both, (ii) at least one pharmaceutically acceptable excipient, and (iii) an amount of the osmotically active composition.

### Therapeutic Agents

Therapeutic agents can include any neurologically active agents acting at synaptic and neuroeffector junction sites. The neurologically active agent useful in the present invention may be one that acts at the synaptic and neuroeffector junctional sites including, but not limited to, a cholinergic agonist, a anticholinesterase agent, catecholamine and other sympathomimetic drugs, an adrenergic receptor antagonist, an antimuscarinic drug, and an agent that acts at the neuromuscular junction and autonomic ganglia.

Examples of suitable cholinergic agonists include, but are not limited to, choline chloride, acetylcholine chloride, methacholine chloride, carbachol chloride, bethanechol chloride, pilocarpine, muscarine, arecoline and the like.

Suitable anticholinesterase agents are exemplified by the group consisting of carbaril, physostigmine, neostigmine, edrophonium, pyridostigmine, demecarium, ambenonium, tetrahydroacridine and the like.

Suitable catecholamines and sympathomimetic drugs include the subclasses of endogenous catecholamines, beta-adrenergic agonists, alpha-adrenergic agonists and other miscellaneous adrenergic agonists.

Within the subclass of endogenous catecholamines, suitable examples include epinephrine, norepinephrine, dopamine and the like. Suitable examples within the subclass of beta-adrenergic agonists include, but are not limited to, isoproterenol, dobutamine, metaproterenol, terbutaline, albuterol, isoetharine, pirbuterol, bitolterol, ritodrine and the like. The subclass of α-adrenergic agonists can be exemplified by methoxamine, phenylephrine, mephentermine, metaraminol, clonidine, guanfacine, guanabenz, methyldopa and the like. Other miscellaneous adrenergic agents include, but are not limited to, amphetamine, methamphetamine, methylphenidate, pemoline, ephedrine and ethylnorepinephrine and the like.

Adrenergic receptor antagonists include the subclasses of α-adrenergic receptor antagonists and β-adrenergic receptor antagonists. Suitable examples of neurologically active agents that can be classified as α-adrenergic receptor antagonists include, but are not limited to, phenoxybenzamine and related haloalkylamines, phentolamine, tolazoline, prazosin and related drugs, ergot alkaloids and the like. Either selective or nonselective β-adrenergic receptor antagonists are suitable for use in the present invention, as are other miscellaneous β-adrenergic receptor antagonists.

Antimuscarinic drugs are exemplified by the group consisting of atropine, scopolamine, homatropine, belladonna, methscopolamine, methantheline, propantheline, ipratropium, cyclopentolate, tropicamide, pirenzepine and the like.

In addition, therapeutic agents that act at the neuromuscular junction and autonomic ganglia are contemplated. Suitable examples of such neurologically active agents that can be classified as agents that act at the neuromuscular junction and autonomic ganglia include, but are not limited to tubocurarine, alcuronium, β-Erythroidine, pancuronium, gallamine, atracurium, decamethonium, succinylcholine, nicotine, labeline, tetramethylammonium, 1,1-dimethyl-4-phenylpiperazinium, hexamethonium, pentolinium, trimethaphan and mecamylamine, and the like.

Therapeutic agents that act on the central nervous system and the peripheral nervous system are also contemplated. Such neurologically active agents can include nonpeptide neurotransmitters, peptide neurotransmitters and neurohormones, proteins associated with membranes of synaptic vessels, neuromodulators, neuromediators, sedative-hypnotics, antiepileptic therapeutic agents, therapeutic agents effective in the treatment of Parkinsonism and other movement disorders, opioid analgesics and antagonists and antipsychotic compounds.

Nonpeptide neurotransmitters include the subclasses of neutral amino acids-such as glycine and gamma-aminobutyric acid and acidic amino acids-such as glutamate, aspartate, and NMDA receptor antagonist-MK801 (Dizocilpine Maleate). Other suitable nonpeptide neurotransmitters are exemplified by acetylcholine and the subclass of monoamines-such as dopamine, norepinephrine, 5-hydroxytryptamine, histamine, and epinephrine.

Neurotransmitters and neurohormones that are neuroactive peptides include the subclasses of hypothalamic-releasing hormones, neurohypophyseal hormones, pituitary peptides, invertebrate peptides, gastrointestinal peptides, atrial naturetic peptide, and other neuroactive peptides.

The subclass of hypothalamic releasing hormones includes as suitable examples, thyrotropin-releasing hormones, gonadotropin-releasing hormone, somatostatins, corticotropin-releasing hormone and growth hormone-releasing hormone.

The subclass of neurohypophyseal hormones is exemplified by agents such as vasopressin, oxytocin, and neurophysins. Likewise the subclass of pituitary peptides is exemplified by the group consisting of adrenocorticotropic hormone, beta-endorphin, alpha-melanocyte-stimulating hormone, prolactin, luteinizing hormone, growth hormone, and thyrotropin.

Suitable invertebrate peptides are exemplified by the group comprising FMRF amide, hydra head activator, proctolin, small cardiac peptides, myomodulins, buccolins, egg-laying hormone and bag cell peptides. The subclass of gastrointestinal peptides includes such therapeutic agents as vasoactive intestinal peptide, cholecystokinin, gastrin, neurotensin, methionine-enkephalin, leucine-enkephalin, insulin and insulin-like growth factors I and II, glucagon, peptide histidine isoleucineamide, bombesin, motilin and secretins.

Suitable examples of other neuroactive peptides include angiotensin II, bradykinin, dynorphin, opiocortins, sleep peptide(s), calcitonin, CGRP (calcitonin gene-related peptide), neuropeptide Y, neuropeptide Yy, galanin, substance K (neurokinin), physalaemin, Kassinin, uperolein, eledoisin and atrial naturetic peptide.

Proteins associated with membranes of synaptic vesicles include the subclasses of calcium-binding proteins and other synaptic vesicle proteins. The subclass of calcium-binding proteins further includes the cytoskeleton-associated proteins-such as caldesmon, annexins, calelectrin (mammalian), calelectrin (torpedo), calpactin I, calpactin complex, calpactin II, endonexin I, endonexin II, protein II, synexin I; and enzyme modulators, such as p65. Other synaptic vesicle proteins include inhibitors of mobilization (such as synapsin Ia,b and synapsin IIa,b), possible fusion proteins such as synaptophysin, and proteins of unknown function such as p29, VAMP-1,2 (synaptobrevin), VAT-1, rab 3A, and rab 3B.

Neuromodulators can be exemplified by the group consisting of CO2 and ammonia, steroids and steroid hormones, adenosine and other purines, and prostaglandins.

Neuromediators can be exemplified by the group consisting of cyclic AMP, cyclic GMP, and cyclic nucleotide-dependent protein phosphorylation reactions.

Sedative-hypnotics can be exemplified by the group consisting of benzodiazepines and buspirone, barbiturates, and miscellaneous sedative-hypnotics.

Suitable antiepileptic drugs can be exemplified by the groups consisting of, but not limited to, hydantoins such as phenytoin, mephenytoin, and ethotoin; anticonvulsant barbiturates such as phenobarbital and mephobarbital; deoxybarbiturates such as primidone; iminostilbenes such as carbamazepine; succinimides such as ethosuximide, methsuximide, and phensuximide; valproic acid; oxazolidinediones such as trimethadione and paramethadione; benzodiazepines and other antiepileptic agents such as phenacemide, acetazolamide, and progabide.

Neurologically active agents that are effective in the treatment of Parkinsonism and other movement disorders include, but are not limited to, dopamine, levodopa, carbidopa, amantadine, baclofen, diazepam, dantrolene, dopaminergic agonists such as apomorphine, ergolines such as bromocriptine, pergolide, and lisuride, and anticholinergic drugs such as benztropine mesylate, trihexyphenidyl hydrochloride, procyclidine hydrochloride, biperiden hydrochloride, ethopropazine hydrochloride, and diphenhydramine hydrochloride.

Suitable opioid analgesics and antagonists can be exemplified by the group consisting of, but not limited to, endogenous opioid peptides such as enkephalins, endorphins, and dynorphins; morphine and related opioids such as levorphanol and congeners; meperidine and congeners such as piperidine, phenylpiperidine, diphenoxylate, loperamide, and fentanyl; methadone and congeners such as methadone and propoxyphene; pentazocine; nalbuphine; butorphanol; buprenorphine; meptazinol; opioid antagonists such as naloxone hydrochloride; and centrally active antitussive agents such as dextromethorphan.

Neurologically active agents that can be used to treat depression, anxiety or psychosis are also contemplated. Suitable antipsychotic compounds include, but are not limited to, phenothiazines, thioxanthenes, dibenzodiazepines, butyrophenones, diphenylbutylpiperidines, indolones, and rauwolfia alkaloids. Mood alteration drugs that are suitable for use in the present invention include, but are not limited to, tricyclic antidepressants (which include tertiary amines and secondary amines), atypical antidepressants, and monoamine oxidase inhibitors. Examples of suitable drugs that are used in the treatment of anxiety include, but are not limited to, benzodiazepines.

A neurologically active therapeutic agent may be a neuroactive protein, such as human and chimeric mouse/human monoclonal antibodies, erythropoietin and G-CSF, orthoclone OKT3, interferon-gamma, interleukin-1 receptors, t-PA (tissue-type plasminogen activator), recombinant streptokinase, superoxide dismutase, tissue factor pathway inhibitor (TFPI). Alternatively, the neurologically active agent may be a neuroactive nonprotein drug, such as neurotransmitter receptors and pharmacological targets in Alzheimer's disease; Design and Synthesis of BMY21502: A Potential Memory and Cognition Enhancing Agent; muscarinic agonists for the central nervous system; serotonic receptors, agents, and actions; thiazole-containing 5-hydroxytryptamine-3 receptor antagonists; acidic amino acids as probes of glutamate receptors and transporters; L-2-(carboxycyclopropyl)glycines; and N-Methyl-D-aspartic acid receptor antagonists.

The invention also contemplates administration of cancer therapies through the any blood-organ barrier, including the BBB. Non-limiting examples of anti-cancer agents and drugs that can be used in combination with one or more compositions and methods of the invention for the treatment of cancer include, but are not limited to, one or more of: 20-epi-1,25 dihydroxyvitamin D3, 4-ipomeanol, 5-ethynyluracil, 9-dihydrotaxol, abiraterone, acivicin, aclarubicin, acodazole hydrochloride, acronine, acylfulvene, adecypenol, adozelesin, aldesleukin, all-tk antagonists, altretamine, ambamustine, ambomycin, ametantrone acetate, amidox, amifostine, aminoglutethimide, aminolevulinic acid, amrubicin, amsacrine, anagrelide, anastrozole, andrographolide, angiogenesis inhibitors, antagonist D, antagonist G, antarelix, anthramycin, anti-dorsalizing morphogenetic protein-1, antiestrogen, antineoplaston, antisense oligonucleotides, aphidicolin glycinate, apoptosis gene modulators, apoptosis regulators, apurinic acid, ARA-CDP-DL-PTBA, arginine deaminase, asparaginase, asperlin, asulacrine, atamestane, atrimustine, axinastatin 1, axinastatin 2, axinastatin 3, azacitidine, azasetron, azatoxin, azatyrosine, azetepa, azotomycin, baccatin III derivatives, balanol, batimastat, benzochlorins, benzodepa, benzoylstaurosporine, beta lactam derivatives, beta-alethine, betaclamycin B, betulinic acid, BFGF inhibitor, bicalutamide, bisantrene, bisantrene hydrochloride, bisaziridinylspermine, bisnafide, bisnafide dimesylate, bistratene A, bizelesin, bleomycin, bleomycin sulfate, BRC/ABL antagonists, breflate, brequinar sodium, bropirimine, budotitane, busulfan, buthionine sulfoximine, cactinomycin, calcipotriol, calphostin C, calusterone, camptothecin derivatives, canarypox IL-2, capecitabine, caracemide, carbetimer, carboplatin, carboxamide-amino-triazole, carboxyamidotriazole, carest M3, carmustine, cam 700, cartilage derived inhibitor, carubicin hydrochloride, carzelesin, casein kinase inhibitors, castanospermine, cecropin B, cedefingol, cetrorelix, chlorambucil, chlorins, chloroquinoxaline sulfonamide, cicaprost, cirolemycin, cisplatin, cis-porphyrin, cladribine, clomifene analogs, clotrimazole, collismycin A, collismycin B, combretastatin A4, combretastatin analog, conagenin, crambescidin 816, crisnatol, crisnatol mesylate, cryptophycin 8, cryptophycin A derivatives, curacin A, cyclopentanthraquinones, cyclophosphamide, cycloplatam, cypemycin, cytarabine, cytarabine ocfosfate, cytolytic factor, cytostatin, dacarbazine, dacliximab, dactinomycin, daunorubicin hydrochloride, decitabine, dehydrodidemnin B, deslorelin, dexifosfamide, dexormaplatin, dexrazoxane, dexverapamil, dezaguanine, dezaguanine mesylate, diaziquone, didemnin B, didox, diethylnorspermine, dihydro-5-azacytidine, dioxamycin, diphenyl spiromustine, docetaxel, docosanol, dolasetron, doxifluridine, doxorubicin, doxorubicin hydrochloride, droloxifene, droloxifene citrate, dromostanolone propionate, dronabinol, duazomycin, duocarmycin SA, ebselen, ecomustinc, edatrexate, edelfosine, edrecolomab, eflornithine, eflornithine hydrochloride, elemene, elsamitrucin, emitefur, enloplatin, enpromate, epipropidine, epirubicin, epirubicin hydrochloride, epristeride, erbulozole, erythrocyte gene therapy vector system, esorubicin hydrochloride, estramustine, estramustine analog, estramustine phosphate sodium, estrogen agonists, estrogen antagonists, etanidazole, etoposide, etoposide phosphate, etoprine, exemestane, fadrozole, fadrozole hydrochloride, fazarabine, fenretinide, filgrastim, finasteride, flavopiridol, flezelastine, floxuridine, fluasterone, fludarabine, fludarabine phosphate, fluorodaunorunicin hydrochloride, fluorouracil, flurocitabine, forfenimex, formestane, fosquidone, fostriecin, fostriecin sodium, fotemustine, gadolinium texaphyrin, gallium nitrate, galocitabine, ganirelix, gelatinase inhibitors, gemcitabine, gemcitabine hydrochloride, glutathione inhibitors, hepsulfam, heregulin, hexamethylene bisacetamide, hydroxyurea, hypericin, ibandronic acid, idarubicin, idarubicin hydrochloride, idoxifene, idramantone, ifosfamide, ilmofosine, ilomastat, imidazoacridones, imiquimod, immunostimulant peptides, insulin-like growth factor-1 receptor inhibitor, interferon agonists, interferon alpha-2A, interferon alpha-2B, interferon alpha-N1, interferon alpha-N3, interferon beta-IA, interferon gamma-IB, interferons, interleukins, iobenguane, iododoxorubicin, iproplatin, irinotecan, irinotecan hydrochloride, iroplact, irsogladine, isobengazole, isohomohalicondrin B, itasetron, jasplakinolide, kahalalide F, lamellarin-N triacetate, lanreotide, lanreotide acetate, leinamycin, lenograstim, lentinan sulfate, leptolstatin, letrozole, leukemia inhibiting factor, leukocyte alpha interferon, leuprolide acetate, leuprolide/estrogen/progesterone, leuprorelin, levamisole, liarozole, liarozole hydrochloride, linear polyamine analog, lipophilic disaccharide peptide, lipophilic platinum compounds, lissoclinamide 7, lobaplatin, lombricine, lometrexol, lometrexol sodium, lomustine, lonidamine, losoxantrone, losoxantrone hydrochloride, lovastatin, loxoribine, lurtotecan, lutetium texaphyrin, lysofylline, lytic peptides, maitansine, mannostatin A, marimastat, masoprocol, maspin, matrilysin inhibitors, matrix metalloproteinase inhibitors, maytansine, mechlorethamine hydrochloride, megestrol acetate, melengestrol acetate, melphalan, menogaril, merbarone, mercaptopurine, meterelin, methioninase, methotrexate, methotrexate sodium, metoclopramide, metoprine, meturedepa, microalgal protein kinase C inhibitors, MIF inhibitor, mifepristone, miltefosine, mirimostim, mismatched double stranded RNA, mitindomide, mitocarcin, mitocromin, mitogillin, mitoguazone, mitolactol, mitomalcin, mitomycin, mitomycin analogs, mitonafide, mitosper, mitotane, mitotoxin fibroblast growth factor-saporin, mitoxantrone, mitoxantrone hydrochloride, mofarotene, molgramostim, monoclonal antibody, human chorionic gonadotrophin, monophosphoryl lipid a/myobacterium cell wall SK, mopidamol, multiple drug resistance gene inhibitor, multiple tumor suppressor 1-based therapy, mustard anticancer agent, mycaperoxide B, mycobacterial cell wall extract, mycophenolic acid, myriaporone, n-acetyldinaline, nafarelin, nagrestip, naloxone/pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, nocodazole, nogalamycin, n-substituted benzamides, O6-benzylguanine, octreotide, okicenone, oligonucleotides, onapristone, ondansetron, oracin, oral cytokine inducer, ormaplatin, osaterone, oxaliplatin, oxaunomycin, oxisuran, paclitaxel, paclitaxel analogs, paclitaxel derivatives, palauamine, palmitoylrhizoxin, pamidronic acid, panaxytriol, panomifene, parabactin, pazelliptine, pegaspargase, peldesine, peliomycin, pentamustine, pentosan polysulfate sodium, pentostatin, pentrozole, peplomycin sulfate, perflubron, perfosfamide, perillyl alcohol, phenazinomycin, phenylacetate, phosphatase inhibitors, picibanil, pilocarpine hydrochloride, pipobroman, piposulfan, pirarubicin, piritrexim, piroxantrone hydrochloride, placetin A, placetin B, plasminogen activator inhibitor, platinum complex, platinum compounds, platinum-triamine complex, plicamycin, plomestane, porfimer sodium, porfiromycin, prednimustine, procarbazine hydrochloride, propyl bis-acridone, prostaglandin J2, prostatic carcinoma antiandrogen, proteasome inhibitors, protein A-based immune modulator, protein kinase C inhibitor, protein tyrosine phosphatase inhibitors, purine nucleoside phosphorylase inhibitors, puromycin, puromycin hydrochloride, purpurins, pyrazofurin, pyrazoloacridine, pyridoxylated hemoglobin polyoxyethylene conjugate, RAF antagonists, raltitrexed, ramosetron, RAS farnesyl protein transferase inhibitors, RAS inhibitors, RAS-GAP inhibitor, retelliptine demethylated, rhenium RE 186 etidronate, rhizoxin, riboprine, ribozymes, RH retinamide, RNAi, rogletimide, rohitukine, romurtide, roquinimex, rubiginone B1, ruboxyl, safingol, safingol hydrochloride, saintopin, sarenu, sarcophytol A, sargramostim, SDI 1 mimetics, semustine, senescence derived inhibitor 1, sense oligonucleotides, signal transduction inhibitors, signal transduction modulators, simtrazene, single chain antigen binding protein, sizofiran, sobuzoxane, sodium borocaptate, sodium phenylacetate, solverol, somatomedin binding protein, sonermin, sparfosate sodium, sparfosic acid, sparsomycin, spicamycin D, spirogermanium hydrochloride, spiromustine, spiroplatin, splenopentin, spongistatin 1, squalamine, stem cell inhibitor, stem-cell division inhibitors, stipiamide, streptonigrin, streptozocin, stromelysin inhibitors, sulfinosine, sulofenur, superactive vasoactive intestinal peptide antagonist, suradista, suramin, swainsonine, synthetic glycosaminoglycans, talisomycin, tallimustine, tamoxifen methiodide, tauromustine, tazarotene, tecogalan sodium, tegafur, tellurapyrylium, telomerase inhibitors, teloxantrone hydrochloride, temoporfin, temozolomide, teniposide, teroxirone, testolactone, tetrachlorodecaoxide, tetrazomine, thaliblastine, thalidomide, thiamiprine, thiocoraline, thioguanine, thiotepa, thrombopoietin, thrombopoietin mimetic, thymalfasin, thymopoietin receptor agonist, thymotrinan, thyroid stimulating hormone, tiazofurin, tin ethyl etiopurpurin, tirapazamine, titanocene dichloride, topotecan hydrochloride, topsentin, toremifene, toremifene citrate, totipotent stem cell factor, translation inhibitors, trestolone acetate, tretinoin, triacetyluridine, triciribine, triciribine phosphate, trimetrexate, trimetrexate glucuronate, triptorelin, tropisetron, tubulozole hydrochloride, turosteride, tyrosine kinase inhibitors, tyrphostins, UBC inhibitors, ubenimex, uracil mustard, uredepa, urogenital sinus-derived growth inhibitory factor, urokinase receptor antagonists, vapreotide, variolin B, velaresol, veramine, verdins, verteporfin, vinblastine sulfate, vincristine sulfate, vindesine, vindesine sulfate, vinepidine sulfate, vinglycinate sulfate, vinleurosine sulfate, vinorelbine, vinorelbine tartrate, vinrosidine sulfate, vinxaltine, vinzolidine sulfate, vitaxin, vorozole, zanoterone, zeniplatin, zilascorb, zinostatin, zinostatin stimalamer, and zorubicin hydrochloride, as well as salts, homologs, analogs, derivatives, enantiomers and/or functionally equivalent compositions thereof.

Other examples of agents useful in the treatment of cancer include, but are not limited to, one or more of Ributaxin, Herceptin, Quadramet, Panorex, IDEC-Y2B8, BEC2, C225, Oncolym, SMART M195, ATRAGEN, Ovarex, Bexxar, LDP-03, ior t6, MDX-210, MDX-11, MDX-22, OV103, 3622W94, anti-VEGF, Zenapax, MDX-220, MDX-447, MELIMMUNE-2, MELIMMUNE-1, CEACIDE, Pretarget, NovoMAb-G2, TNT, Gliomab-H, GNI-250, EMD-72000, LymphoCide, CMA 676, Monopharm-C, 4B5, ior egf r3, ior c5, BABS, anti-FLK-2, MDX-260, ANA Ab, SMART 1D10 Ab, SMART ABL 364 Ab and ImmuRAIT-CEA.

The method described herein also contemplates the enhanced ability to deliver therapeutic antibodies to a subject across the blood-brain barrier. Antibodies can be prepared from the intact polypeptide or fragments containing peptides of interest as the immunizing agent. A preferred antigenic polypeptide fragment is 15-100 contiguous amino acids of protein antigen of interest. In one embodiment, the peptide is located in a non-transmembrane domain of the polypeptide, e.g., in an extracellular or intracellular domain. An exemplary antibody or antibody fragment binds to an epitope that is accessible from the extracellular milieu and that alters the functionality of the protein. In certain embodiments, the antibodies contemplated recognize and are specific for one or more epitopes of a protein antigen of interest. A therapeutically useful antibody includes a “humanized” monoclonal antibody. Humanized monoclonal antibodies are produced by transferring mouse complementarity determining regions from heavy and light variable chains of the mouse immunoglobulin into a human variable domain, then substituting human residues into the framework regions of the murine counterparts. The use of antibody components derived from humanized monoclonal antibodies obviates potential problems associated with immunogenicity of murine constant regions. A therapeutically effective amount of an antibody as disclosed herein relates generally to the amount needed to achieve a therapeutic objective. This may be a binding interaction between the antibody and its target antigen that, in certain cases, interferes with the functioning of the target, and in other cases, promotes a physiological response. The amount required to be administered will furthermore depend on the binding affinity of the antibody for its specific antigen, and will also depend on the rate at which an administered antibody is depleted from the free volume of the subject to which it is administered. The dosage ranges for the administration of monoclonal antibodies are large enough to produce the desired effect, and will vary with age, condition, weight, sex, age and the extent of the condition to be treated, and can readily be determined by one skilled in the art. Common ranges for therapeutically effective dosing of an antibody or antibody fragment may be, by way of nonlimiting example, from about 0.1 mg/kg body weight to about 2000 mg/kg body weight. Common dosing frequencies may range, for example, from twice daily to once a week. The monoclonal antibodies can be administered intravenously, intraperitoneally, intramuscularly, and/or subcutaneously.

The method also relates to the administration of immunoconjugates comprising an antibody conjugated to a chemical agent, or a radioactive isotope (i.e., a radioconjugate) for administration to a portion of an organ, e.g., brain tissue, using the methods of the invention. Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. Sec WO94/11026.

In some embodiments, the at least one therapeutic agent can be a small molecule therapeutic agent. In other embodiments, the therapeutic agent can be a non-small molecule therapeutic agent, such as a biologic. Therapeutic agents can include, but are not limited to, gene therapy agents, proteins (including, but not limited to, natural proteins, recombinant therapeutic proteins, antibodies, and the like), sugars, nucleic acids (including, but not limited to, natural nucleic acids, recombinant nucleic acids, vectors, and the like) and combinations of the same. Vectors can include, but are not limited to, plasmids, viral vectors, cosmids, and artificial chromosomes. In some embodiments, the therapeutic agent can include a polynucleotide. In some embodiments, the therapeutic agent can include a recombinant polynucleotide. In some embodiments, the therapeutic agent can include a vector comprising a recombinant polynucleotide. Viral vectors can include, but are not limited to, recombinant retroviruses, recombinant lentiviruses, recombinant adenoviruses, and recombinant adeno-associated viruses. In some embodiments the therapeutic agent can specifically include an adeno-associated virus (AAV) vector. While not intending to be bound by theory, it is believed that AAV vectors can be beneficial because AAV is highly efficient at transducing CNS cells, AAV vectors have demonstrated long-term (>8 years) expression in the CNS, and AAV vectors have a small viral capsids with a diameter of 20 to 25 nanometers allowing better passage within the brain extracellular space. In addition, wild-type AAV is not known to cause disease and the transgene delivered from an AAV vector generally does not integrate into the host genome, which reduces concern of carcinogenesis. In some embodiments, the therapeutic agent can include at least one selected from the group of proteins (including recombinant proteins and antibodies), anti-sense RNA, siRNA, mRNA, and RNAi. In some embodiments, the therapeutic agent comprises an antibody.

It should be appreciated by the person skilled in the art that the at least one therapeutic agent may be present as a pharmaceutically acceptable salt, a pharmaceutically acceptable solvate, or as a prodrug.

In some embodiments, the osmotically active composition nor the formulation comprising the therapeutic agent, at least one contrast agent, or both, are intended to be delivered as an aerosol or nasally. In some embodiments, the osmotically active composition nor the formulation comprising the therapeutic agent, at least one contrast agent, or both, are intended to target the P2X3, TRPV, or ACE2 receptors. In some embodiments, the osmotically active composition and the formulation comprising the therapeutic agent, at least one contrast agent, or both, are substantially devoid of neuronal agonists (e.g., capsaicin, dihydrocapsaicin, nordihydrocapsaicin, homocapsaicin, homodihydrocapsaicin, nonivamide, capsiate, dihydrocapsiate, nordihydrocapsiate, arvanil, olvanil, menthol, isomenthol, carveol, terpineols, pulegol, isopulolegol, hinokitiol, myrtenol, and verbenol). In some embodiments, the osmotically active composition and the formulation comprising the therapeutic agent, at least one contrast agent, or both, are substantially devoid of gelling agents. In some embodiments, the osmotically active composition and the formulation comprising the therapeutic agent, at least one contrast agent, or both, are substantially devoid of cryoprotectants (e.g., compositions comprising DMSO). In some embodiments, the osmotically active composition nor the formulation comprising the therapeutic agent, at least one contrast agent, or both, are intended for use with arthroscopic surgeries. In some embodiments, the osmotically active composition and the formulation comprising the therapeutic agent, at least one contrast agent, or both, are substantially devoid of contrast agent. In some embodiments, the disruption of the BBB is not performed using non-invasive MR (magnetic resonance) imaging with a contrast agent to visualize local parenchymal transcatheter perfusion. In some embodiments, the formulation is never administered intravenously.

### Other Methods of Treating

In a second aspect, a method of treating cancer, inflammatory diseases, genetic diseases, or a neurological disease in a subject is disclosed, said method comprising:
administering an effective amount of an osmotically active composition to the subject to disrupt a blood-organ barrier at an organ to be treated; and
administering a therapeutically effective amount of a formulation comprising at least one therapeutic agent to the subject,
   wherein the osmotically active composition induces transient permeabilization of the blood-organ barrier to enhance delivery of the at least one therapeutic agent to the organ of the subject, thereby treating said disease.

In some embodiments of the second aspect, the method of treating cancer, inflammatory diseases, genetic diseases, or a neurological disease in a subject comprises:
administering an effective amount of an osmotically active composition to the subject to disrupt a blood-organ barrier at an organ to be treated; and
ceasing the administration of the osmotically active composition to the subject for a time x; and then
immediately a therapeutically effective amount of a formulation comprising at least one therapeutic agent to the subject,
   wherein the osmotically active composition induces transient permeabilization of the blood-organ barrier to enhance delivery of the at least one therapeutic agent to the organ of the subject, thereby treating said disease.

In some embodiments, the blood-organ barrier is the BBB and the organ is a brain. In some embodiments, the osmotically active composition is administered intraarterially. In some embodiments, the osmotically active composition is administered intraarterially via a catheter placed in an artery that is directly feeding the organ or a portion thereof. In some embodiments, the formulation comprising at least one therapeutic agent may be administered by intraarterial infusion. In some embodiments, the formulation is administered intraarterially via a catheter placed in an artery that is directly feeding the organ or a portion thereof. In some embodiments, the formulation is administered, e.g., intraarterially, such that the formulation substantially displaces the blood in the artery and downstream capillary vessels. In some embodiments, the time x between the stopping of the administration of the osmotically active composition to the subject and the administration of the formulation comprising at least one therapeutic agent to the subject is in a range from about 1 sec to about 30 min, or about 1 sec to 5 min, or about 1 sec to about 1 min, or about 1 sec to about 30 sec. In some embodiments, the same or a different catheter is used to administer the osmotically active composition and the formulation comprising the at least one therapeutic agent. In some embodiments, the at least one therapeutic agent is intended to pass through the subject's blood-organ barrier, e.g., BBB, to treat a disease such as the cancer, e.g., brain cancer or head and neck cancer, an inflammatory disease, a genetic disease, a neurological disease, a mental disease or any other disease associated with an organ. In some embodiments, the cancer is brain cancer or cancers that have metastasized to the brain. In some embodiments, the cancer is body cancer.

In a third aspect, an article is described that can be attached to a catheter line, for example using a Luer lock or the equivalent thereof, wherein the article comprises a first compartment for a first bolus of the osmotically active composition and a second compartment for a second bolus of the formulation comprising at least one therapeutic agent, at least one contrast agent, or both. Upon attachment of the article to the catheter line, the healthcare professional will administer the boluses to the organ via the catheter. The article is arranged to ensure that the first bolus comprising the osmotically active composition first enters the catheter for delivery to the blood-organ barrier of the organ. After the delivery of the first bolus, the second bolus comprising the formulation comprising at least one therapeutic agent, at least one contrast agent, or both, enters the catheter for delivery of the at least one therapeutic agent to the organ. It should be appreciated that the first and second boluses are a known amount of the respective compositions. In some embodiments, the administration of the second bolus occurs immediately after completion of the administration of the first bolus, i.e., time x=0 sec. In some other embodiments, the administration of the second bolus occurs at a time x after completion of the administration of the first bolus, as described herein. It should be appreciated that the article can be engineered to deliver the first and second boluses simultaneously as well.

The features and advantages of the invention are more fully shown by the illustrative examples discussed below.

### EXAMPLE 1

### Study Design

This example focused on evaluating the effectiveness of intra-arterial hyperosmotic agents in inducing transient permeabilization of the blood-brain barrier (BBB) in mice to enhance drug delivery into the brain parenchyma. The outcomes achieved with the previously established method of OBBBO using intraarterial (IA) 25% mannitol injection were compared with a new method of OBBBO using IA injection of 4% sodium chloride (NaCl) in 25% mannitol. It was hypothesized that this increase in osmotic power should enhance the BBBO and promote more effective drug delivery to brain tissue.

Towards that end, the distribution of Gadolinium (Gd) within the brain parenchyma using magnetic resonance imaging (MRI) after OBBBO with 25% mannitol with and without 4% NaCl was evaluated. Then, monoclonal antibody (mAb) bevacizumab was conjugated to deferoxamine (DFO) and the conjugate was radiolabeled with zirconium-89 (⁸⁹Zr). OBBBO was performed using 25% mannitol or 4% NaCl in 25% mannitol, followed by intraarterial (IA) injection of the radiolabeled mAb and positron-emission tomography (PET) imaging to assess the distribution of the mAb. The percentages of injected dose per cubic centimeter of tissue (% ID/g) and standardized uptake values (SUV) were calculated for selected organs and regions.

### Materials

All chemicals were purchased from Sigma-Aldrich or Fisher Scientific unless otherwise specified. Mannitol (Hospira, Inc., Lake Forest, IL; NDC 0409-4031-16) was used for OBBBO and for preparation of 4% saline in 25% mannitol solution. Sodium chloride (Sigma-Aldrich, Saint Louis, MO; 1613804-1G) was diluted in mannitol to reach 4% of concentration and filtered with 0.22 μm pore membrane. Bevacizumab (Avastin [Roche]; 4 mL, 25 mg/mL) was obtained from the University of Maryland Medical Center Pharmacy. ⁸⁹Zr(C204) 2 (half-life, 78.4 h) and 1-(4-isothiocyanatophenyl)-3-[6, 17-dihydroxy-7,10,18,21-tetraoxo-27-(N-acetylhydroxylamino)-6,11,17,22-tetraazaheptaeicosine] thiourea (p-SCN-Bn-deferoxamine, catalog number B-705) were obtained from 3D Imaging and Macrocyclics, respectively. All reagents and solvents were used as received, without further purification.

### Methods

a. Synthesis of Bevacizumab-DFO (BVDFO) Conjugate

For conjugation with DFO, 20 mg of bevacizumab was purified using ultrafiltration with Millipore Amicon Ultra Centrifugal Filters 50K (catalog number VV-29969-76). After purification, bevacizumab was reconstituted in 1 mL of saline and pH was adjusted to 9 using 0.1 M Na₂CO₃. A 4-fold molar equivalent of SCN-Bn-deferoxamine dissolved in dimethyl sulfoxide (DMSO) was added, and conjugation was performed for 30 min at 37° C. in a thermomixer at 550 rpm. The resulting BVDFO conjugate was purified using 7MWCO Zeba Spin Desalting Column (ThermoFisher, Cat. #89889) and stored at −20° C. until further use. The protein concentration in the purified BVDFO conjugate was determined using NanoDrop ND-1000 Spectrophotometer by absorbance at 280 nm.

b. Radiolabeling of BVDFO

All procedures involving handling of radioactive materials followed institutional Environmental Health and Safety (EHS), Nuclear Regulatory Commission (NRC), and ALARA (as low as reasonably achievable) protocols to minimize personnel exposure to emitted radiation.

BVDFO was radiolabeled with ⁸⁹Zr using a modification of a previously reported procedure [Vosjan, 2010]. Briefly, 2-3 mCi of ⁸⁹Zr-oxalate was suspended in 1 M oxalic acid to get 100 uL total volume of the solution. Then, 50 uL 2 M Na₂CO₃ was added. The solution was incubated for 3 minutes at room temperature while lightly shaking. Then, 150 uL 1 M HEPES, 355 uL BVDFO and 350 uL 1 M HEPES were successively added to the solution. The pH was adjusted to 6.8-7 as needed using 0.1 M Na₂CO₃. The reaction was incubated for 1 hour while shaking. Then, the reaction mixture was purified using 7K MWCO Zeba Spin Desalting Column. Instant thin-layer chromatography (iTLC) was preformed both for crude reaction mixture and purified solution using 50 mM diethylenetriaminepentaacetic acid (DTPA) as a mobile phase. High-performance liquid chromatography (HPLC) with radio flowmetry was performed using Agilent 1260 Infinity II HPLC system and Waters XBridge Protein BEH SEC column, directly coupled with Elysia Raytest GABI Nova radio flow meter. 0.1 M phosphate buffer (pH 6.4) was used as a mobile phase at a flow rate of 800 uL/min. The ⁸⁹Zr-bevacizumab (⁸⁹Zr-BVDFO) conjugate was used for injections either immediately or the next day after overnight storage in +4° C.

c. Intraarterial Catheterization

All procedures were approved by the Institutional Animal Care and Use Committee (IACUC) at the University of Maryland, Baltimore. Male C57BL6 mice (10-12 weeks of age, 25-30 g, University of Maryland, Baltimore) were anesthetized with 1.5-2% isoflurane. For the catheterization via the common carotid artery (CCA), the bifurcation of CCA was exposed, and the pterygopalatine artery (PPA) and occipital artery (OA) were coagulated. The external carotid artery (ECA) was temporarily ligated with a 7-0 silk suture to route the entire flow into the internal carotid artery (ICA). A temporary ligature was placed on the carotid bifurcation and the proximal CCA was permanently ligated. A small arteriotomy was made between ligations. A microcatheter (PE-8-100, SAI Infusion Technologies) was flushed with 1% heparin (500 units per ml, heparin sodium, Upjohn), inserted into the CCA and tightly ligated to the vessel and release ligature on the carotid bifurcation. After OBBBO minute-long infusion of 25% mannitol or 4% saline in 25% mannitol at a speed of 0.15 mL/min.

d. MR Imaging

T1 scans provide the best contrast visible as hyperintensity for paramagnetic contrast agents such as Gd-containing compounds. After hyperosmotic treatment, T1-weighted images were acquired to verify BBB status. T2-weighted scans to evaluate the brain injury. To evaluate the safety, mice were assessed for neuropathological sequelae using MRI (days 3 and 7) and histology (day 7).

e. Histology and Immunohistochemistry

To evaluate the temporary effect of OBBBO the locations of BBBO can be revealed with immunohistochemistry by using an antibody against the animal's own IgG. Areas with “leaky” BBB allow serum proteins to pass into the brain parenchyma, e.g., IgG's and albumin. Anti-IgG Stain was performed after OBBBO and at 3 days post-OBBBO. To evaluate the safety of OBBBO on day 7 after OBBBO, animals were transcardially perfused with 5% sucrose and then with 4% paraformaldehyde. The brains were cryopreserved in 30% sucrose and cryosectioned at 30-μm. Hematoxylin and Eosin (H&E) staining to examine the overall tissue morphology and identify any signs of structural damage or inflammation. Primary antibodies and dilutions were used as follows: anti-GFAP (1:250, Dako); anti-Ibal (1:250, Wako); and anti-NeuN (1:100, Cell Signaling Technology). GFAP staining helps in identifying the presence and distribution of astrocytes, activation in response to injury, inflammation, and degree of gliosis. NeuN staining enabled the evaluation of neuronal density and health, while IBA1 staining was employed to detect microglial activation, providing insights into potential neuroinflammatory responses. The secondary antibody was goat anti-rabbit (Alexa Fluor-488, 1:200, Molecular Probes).

f. PET/CT Imaging

Approximately 7.5 MBq (˜202 μCi) of ⁸⁹Zr-BVDFO reconstituted in 0.2 mL of saline was infused intraarterially at a speed of 0.15 mL/min and each animal was transferred to the PET/CT scanner. Whole-body PET/CT imaging was performed immediately after infusion using Siemens Inveon PET-CT Scanner (Siemens Healthineers). A CT scan was performed after the PET scan for anatomic co-registration. PET data was reconstructed and corrected for dead time and radioactive decay. The presented PET/CT images were generated using Amira 3D (ThermoFisher). Regions of interest (ROIs), including right and left brain hemispheres, heart, lungs, and liver, were demarcated in the axial plane, and standardized uptake values (SUV) were calculated using the Fiji ImageJ PET/CT plugin.

g. Ex Vivo Biodistribution of ⁸⁹Zr-BVDFO

On completion of PET/CT after infusion of ⁸⁹Zr-BVDFO, the mice were euthanized and left and right brain hemispheres, heart, lungs, liver, spleen, kidneys, and blood were harvested, weighed using Mettler Toledo ME103TE Balance and radioactivity of each was measured using CRC®-55tR Dose Calibrator and Well Counter, Mirion Technologies. To calculate the percentage injected dose per cubic centimeter of tissue (% ID/g), first, the radioactivity present in the syringe after injection was measured and subtracted from initial measurement before injection to calculate the total injected dose. Then, the percentage of each organ's radioactivity measurement relative to the total injected dose was calculated and divided by the weight of the organ. The biodistribution data shown are mean % ID/g+SE.

### Statistical Analysis

If R is used: R 4.3.1 was used for statistical analysis. Linear mixed modeling with restricted maximum likelihood (REML) method was performed using ‘Imer’ package, with % ID/g or SUVmean or SUVmax as a dependent variable, organs and BBBO type as fixed effects and mouse ID as a random effect. Estimated marginal means were calculated using ‘emmeans’ package for organ and BBBO interactions, and p-values for pairwise comparisons were extracted.

If SAS is used: PROC MIXED (SAS, 9.04) was used for statistical analysis, with the lowest-mean-square test for comparisons between groups. The terms repeated and random were used for repeated measures and to express random effects, respectively.

### Results

a. Safety and Long-Term Consequences of OBBBO

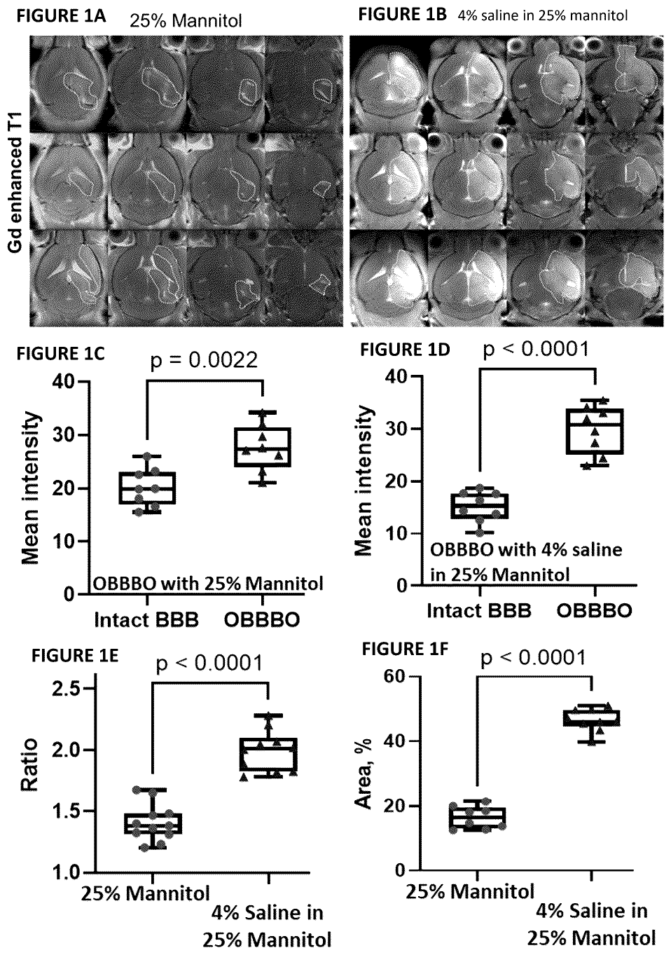

Initial calculations were performed to generate signal change maps depicting contrast accumulation on a T1-weighted scan after OBBBO. This approach effectively demonstrated the impact of Blood-Brain Barrier Opening (BBBO), as evidenced by the enhanced Gd signal on the T1-weighted scan (FIGS. 1A-1B). By assessing the intensity of Gadolinium enhancement, which regions of brain tissue have experienced BBBO can be determined. Subsequently, the area of these affected regions can be calculated and comparative analyses conducted to discern variations in the extent of BBBO across different hyperosmotic agents. In both groups, comparisons of intensity have shown significant differences between BBBO area and intact BBB (FIGS. 1C-1D). Comparisons of ratio of OBBBO area versus BBB intact area between BBBO with 25% mannitol or 4% saline in 25% mannitol shows significant differences preferring the second osmotic agent (FIG. 1E). This approach allowed for the quantitative evaluation and comparison between the areas of the brain where BBBO has been induced (FIG. 1F). In OBBBO by IA 4% saline in 25% mannitol showed an average signal change area of 41.64±7.8% (n=8), which is significantly greater (p>0.0001) (FIG. 1F) than in the case of OBBBO by 25% mannitol (16.74±4.9% (n=8)).

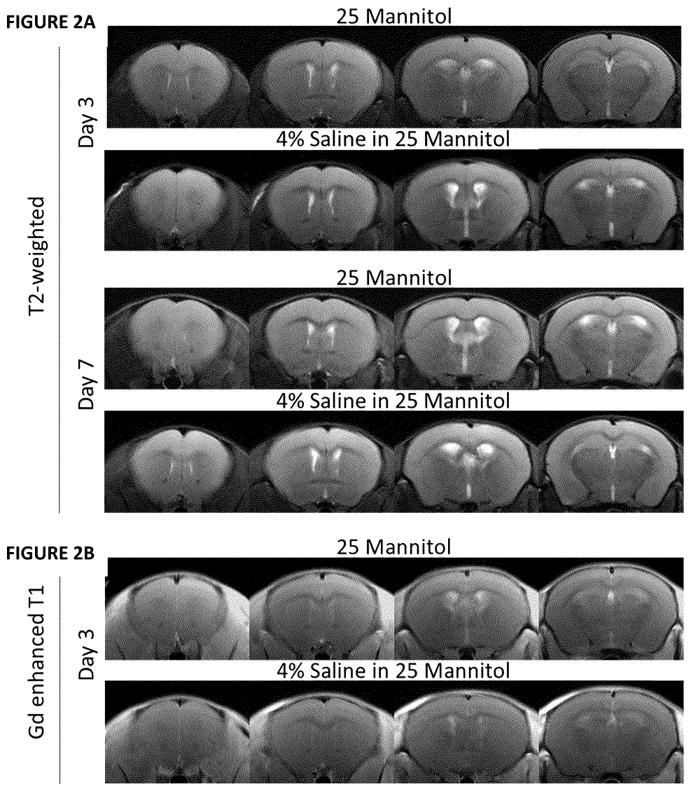

To evaluate the safety, mice were assessed for neuropathological sequelae using MRI and histology. Turbo RARE MRI showed no abnormalities 3 and 7 days post-OBBBO, suggesting a lack of edema or inflammation and microhemorrhages in both groups of animals (FIG. 2A). No T1 Gd enhancement was observed (FIG. 2B), indicating that the BBB reverted. All data revealed that the procedure was safe and did not cause permanent brain damage.

Histological appearance further confirmed these observations. No discernible signs of injury or neuroinflammation were evident upon careful examination of OBBBO region subjected to hematoxylin and eosin staining (data not shown). Glial fibrillary acidic protein (GFAP) and ionized calcium-binding adaptor molecule 1 (IBA-1) staining 7 days post-BBBO showed no elevated astrocytic or microglia activation (GFAP+astrocyte and IBA-1+microglia) in the OBBBO region (data not shown). There was no statistically significant difference in cell density between the OBBBO region and the corresponding contralateral region (data not shown). Similarly, analysis of neuronal nuclei (NeuN) staining indicated no evidence of neuronal loss post-BBBO (data not shown).

### b. ⁸⁹Zr-BVDFO Conjugate Preparation

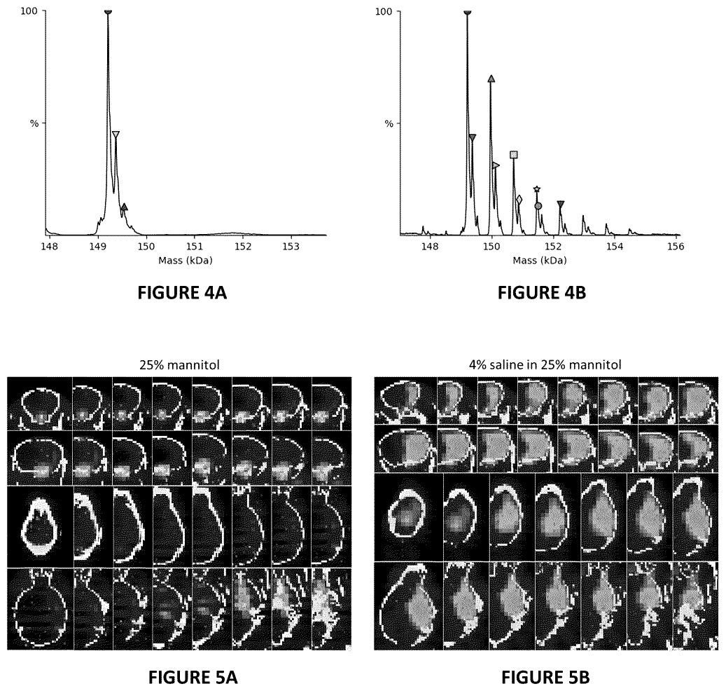

Bevacizumab conjugation with DFO resulted in the binding of 1-3 molecules of deferoxamine per molecule of antibody as revealed by mass spectrometry (FIGS. 4A-4B). Subsequent ⁸⁹Zr chelation was confirmed using iTLC with 98.9% radiolabeling efficiency of the crude reaction and 99.3% radiochemical purity after purification. ⁸⁹Zr-BVDFO was prepared at 5.5±2.0 MBq/mg specific activity.

c. PET Imaging

PET imaging revealed that intra-arterial (IA) injection of ⁸⁹Zr-BVDFO following Osmotic Blood-Brain Barrier Opening (OBBBO) resulted in a substantial accumulation of radioactivity in the ipsilateral hemisphere. FIGS. 5A-5B compare the 25% mannitol group, wherein only a small amount of Gd within the brain parenchyma was observed, to the 4% saline in 25% mannitol, wherein the area and intensity signifying the amount of Gd in the brain parenchyma is much higher.

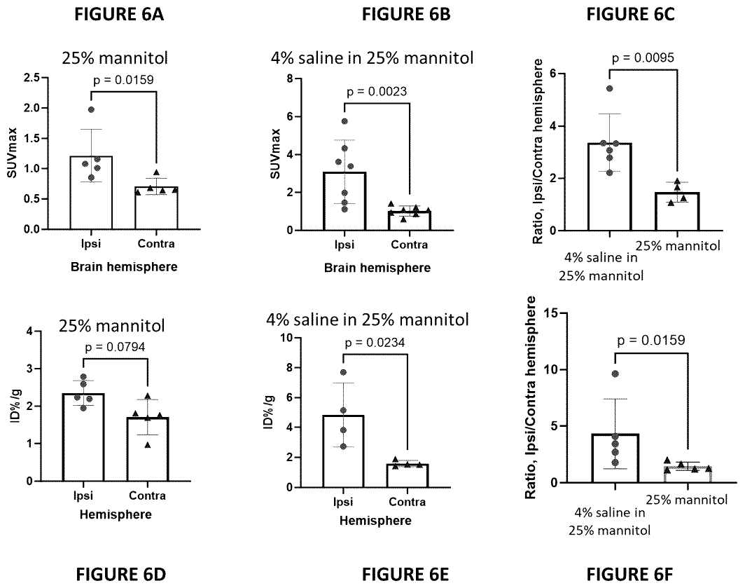

Comparing the SUVmax in the BBBO with 25% mannitol group (FIG. 6A), it can be seen that significant differences were observed in favor of the ipsilateral over the contralateral hemisphere (p=0.0159). This was also true of the 4% saline in 25% mannitol group (FIG. 6B) (p=0.0023), but the standardized uptake value in this group was about three times higher than the 25% mannitol group. Comparing the ipsilateral vs contralateral hemisphere ratio of the groups (FIG. 6C), it can be seen that the 4% saline in 25% mannitol group was significantly greater than 25% mannitol group (p=0.0095).

d. Ex Vivo Biodistribution

To validate the PET/CT imaging results, ⁸⁹Zr-BVDFO was further evaluated in an ex vivo biodistribution analysis. Significantly higher uptake (up to 4 times) in the ipsilateral hemisphere was observed, relative to the contralateral hemisphere, in the group of OBBBO with 4% saline in 25% mannitol (FIG. 6E) while there was no significant statistical difference between the two in the 25% mannitol group (FIG. 6E). Comparing the ipsilateral vs contralateral hemisphere ratio of the groups (FIG. 6E), it can be seen that the 4% saline in 25% mannitol group was significantly greater than 25% mannitol group (p=0.0095). In agreement with earlier studies, high uptake was detected in the blood, lungs, spleen, liver, and kidneys.

### Conclusion

In conclusion, osmotic blood-brain barrier opening represents a groundbreaking approach to drug delivery for brain cancer treatment. Addressing BBB permeability challenges and providing a safer, targeted delivery method offers hope for improved outcomes and enhanced patient quality of life. Ongoing research and clinical advancements in this field hold the potential to revolutionize brain cancer and neurological disorder treatment, moving us closer to more effective, less invasive options.

### EXAMPLE 2

### Introduction

Osmotic blood-brain barrier opening (OBBBO) using 25% mannitol has been effective in large animals and patients. However, in mouse models it has been plagued by inconsistent and spatially restricted BBB opening. This example tests the feasibility of improving BBB opening by augmenting osmotic power through the addition of 4% saline to the 25% mannitol solution.

### Methods

A catheter was placed into the right internal carotid artery (ICA) in 14 male C57BL6 mice for intra-arterial (IA) injections. Osmotic BBB opening (OBBBO) was achieved by administering 150 μl of 25% mannitol+4% NaCl over 60 sec via the IA route. To confirm OBBBO, Gadolinium-enhanced T1 MRI was conducted, and safety was confirmed through subsequent serial MRI over 1-2 weeks. The efficacy of this method for delivering antibodies to the brain was demonstrated through PET imaging of radiolabeled antibodies (⁸⁹Zr-bevacizumab), with ex vivo biodistribution studies to confirm biodistribution.

### Results

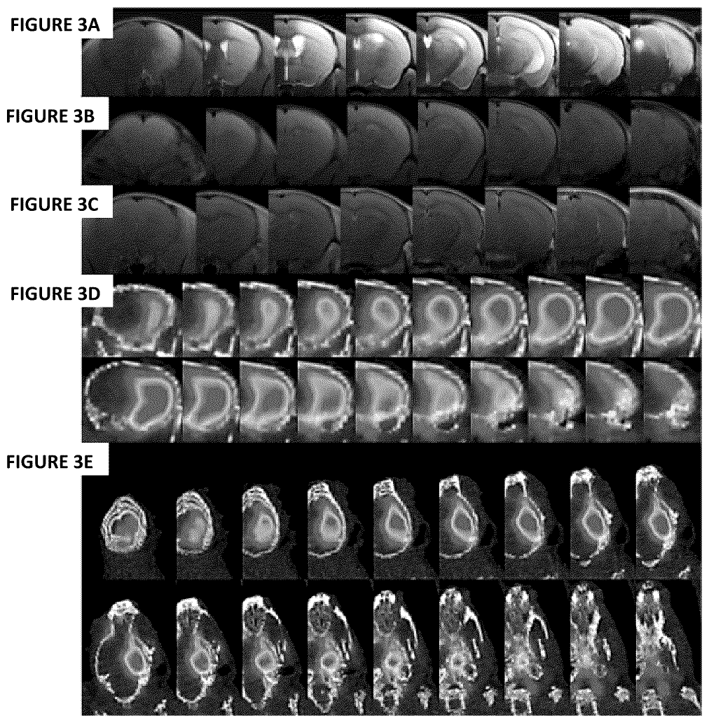

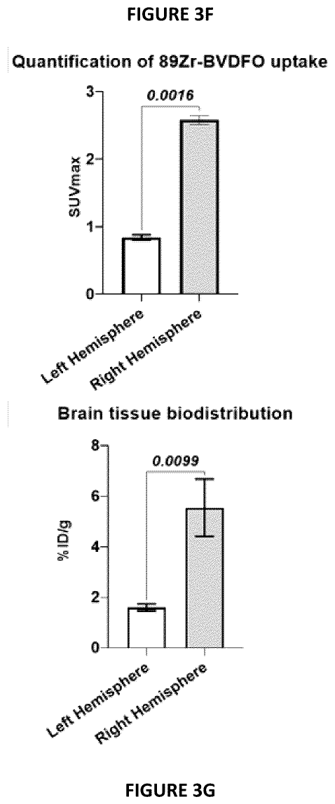

Contrast-enhanced MRI revealed successful OBBBO throughout the entire targeted hemisphere (FIG. 3A), which is a significant improvement over the previous reports of using 25% mannitol alone. Serial MRI on days 3 (FIG. 3B) and 14 (FIG. 3C) post-OBBBO revealed no abnormalities, and no apparent neurological deficits were observed. Standardized uptake values (SUV) demonstrated substantial accumulation of ⁸⁹Zr-bevacizumab (⁸⁹Zr-BVDFO) in the ipsilateral hemisphere on PET images (FIGS. 3D-3F), confirmed by ex vivo biodistribution (FIG. 3G).

### Conclusions

This study demonstrates that doubling osmotic power is a safe and effective strategy to boost OBBBO and antibody delivery to the brain in mice and should be considered for large animal studies and clinical translation.

### EXAMPLE 3

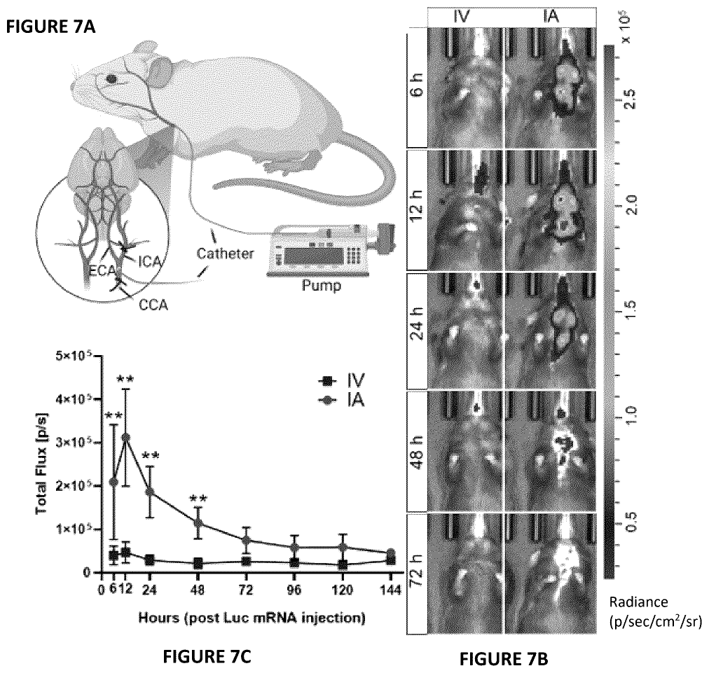

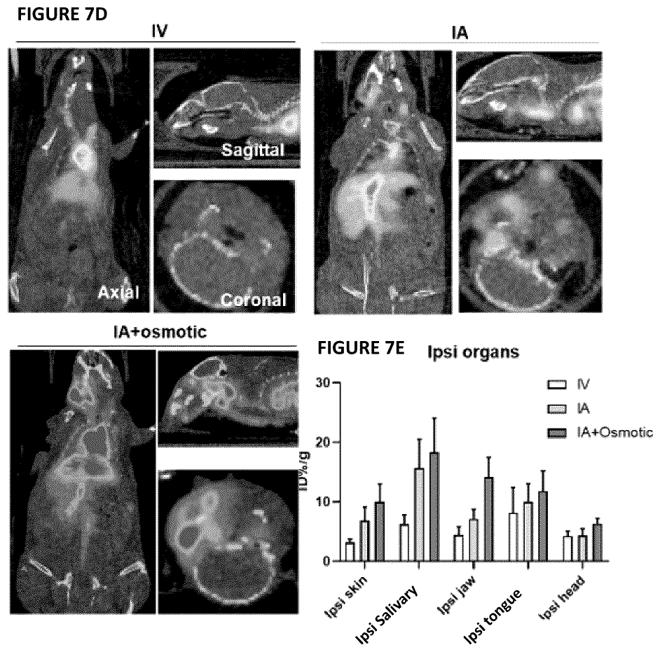

Intra-arterial Administration of mRNA to Peripheral Soft Tissues (Neck Region) mRNA encoding firefly luciferase was used as a surrogate therapeutic agent, enabling easy detection of transgene expression. Bioluminescence imaging (BLI) detected Luc expression in mice that received intra-arterial (IA) administration of Luc mRNA via the external carotid artery, whereas no expression was observed in mice following intravenous (IV) injection (FIG. 7a, b). Quantitative analysis of the BLI signal revealed that Luc expression peaked 12 hours post-injection and gradually declined over the next 72 hours (FIG. 7c).

### Osmotic-Enhanced Intra-Arterial Administration of Monoclonal Antibody (Bevacizumab) to Peripheral Soft Tissues (Neck Region)

Radiolabeled bevacizumab (Zr 89-bevacizumab) was administered via both IA and IV routes. PET imaging demonstrated significantly higher antibody uptake in the head and neck region following IA administration compared to IV injection (FIG. 7d). Ex vivo measurements of drug biodistribution across various organs further corroborated these findings, quantifying radioactivity in specific tissues such as the skin, salivary glands, tongue, and jaw muscle (FIG. 7e). The method comprised pretreatment with composition comprising a hyperosmotic agent, stopping the pretreatment administration, and then administrating the radiolabeled bevacizumab. Without being bound by theory, it is believed that to enhance drug delivery, the pretreatment with the hyperosmotic agent would extract water from the tissue (local dehydration), followed by rapid rehydration upon cessation of the hyperosmotic infusion. This process was expected to improve the transition of the intra-arterially administered drug from the vascular compartment to the parenchymal tissues, thereby increasing drug accumulation in the targeted areas. For the experiment, the osmotically active composition comprised 25% mannitol and 4% NaCl, which was previously established as an effective and safe blood-brain barrier opening agent. Both PET imaging and ex vivo biodistribution analysis revealed that this osmotic treatment significantly enhanced the uptake of radiolabeled antibodies in the targeted head and neck region (FIG. 7d, e).

### Conclusion

IA administration is superior to the IV route for delivering mRNA and antibodies to the head and neck region if the same total dose is administered. Furthermore, osmotic pretreatment can further enhance antibody uptake. These experiments provide compelling evidence for the utility of this strategy in treating various diseases of peripheral tissues, including targeted gene therapy and antibody-based therapies. This approach can also be applied to other therapeutic agents such as small molecules, oligonucleotides, or therapeutic cells. Potential applications include treating cancers, inflammatory, or degenerative disorders across different parts of the body.

### EXAMPLE 4

As described herein, the BBB presents a major obstacle to delivering most therapeutic agents, including the most frequent biological drug antibodies to the CNS. In example 3, it was demonstrated that IA drug administration is more effective than IV after OBBBO if the same total dose is administered. However, it was unclear whether it depended on the higher concentration of antibodies in cerebral circulation after intra-arterial delivery or the inhibitory role of blood post-intravenous administration.

In this example, after the OBBBO opening using a combination of 25% mannitol and 4% NaCl, ⁸⁹Zr-labeled antibodies (bevacizumab) were administered via IV at a 10× higher concentration than IA administration of the same antibodies to adjust for blood dilution, so the same concentration reached cerebral circulation. In experiment 2, IA injections of antibodies suspended in 0.9% NaCl were comparted to IA injections of antibodies suspended in serum. PET imaging and post-mortem antibody biodistribution were used as outcome measures.

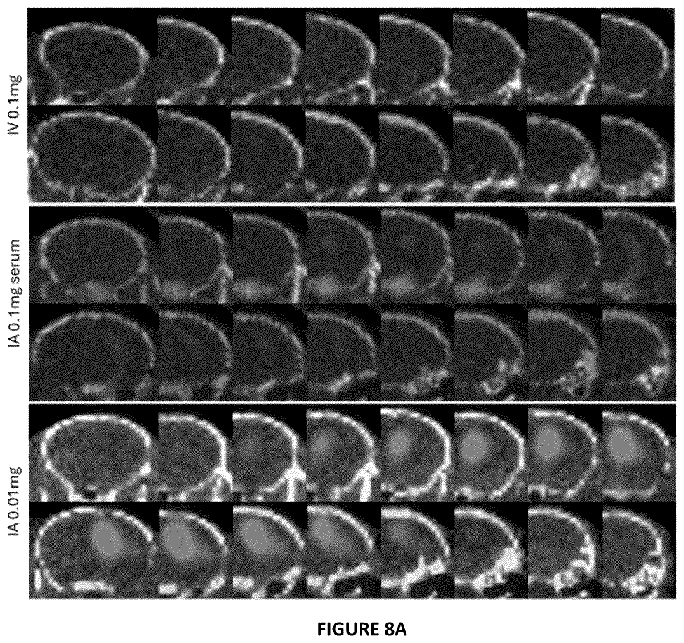

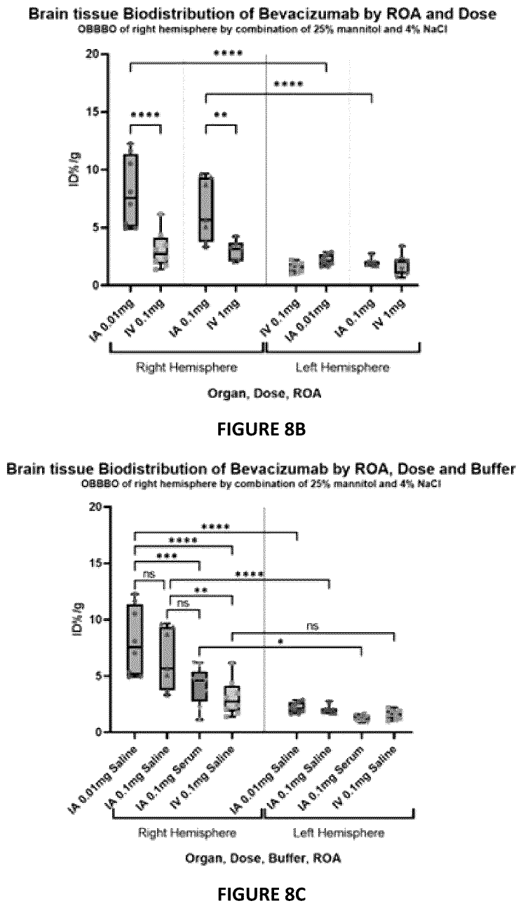

The results verified that IA delivery secured much higher antibody penetration to the brain than IV, even if the same calculated antibody concentration was in the cerebral circulation (the IV total dose was 10× higher than the IA dose). Moreover, CNS penetration of antibodies suspended in 0.9% NaCl was much higher than CNS penetration of antibodies suspended in serum (see, FIGS. 8A-8C).

In conclusion, blood displacement during IA drug delivery might be behind more effective antibody penetration to the brain, while antibodies' contact with the blood may limit their extravasation to the CNS.

Although the invention has been variously disclosed herein with reference to illustrative embodiments and features, it will be appreciated that the embodiments and features described hereinabove are not intended to limit the invention, and that other variations, modifications and other embodiments will suggest themselves to those of ordinary skill in the art, based on the disclosure herein. The invention therefore is to be broadly construed, as encompassing all such variations, modifications and alternative embodiments within the spirit and scope of the claims hereafter set forth.

### REFERENCES

Abbott N J (2013) Blood-brain barrier structure and function and the challenges for CNS drug delivery. J Inherit Metab Dis. 36 (3): 437-49.
Burks, S R, et al., (2021) Blood-brain barrier opening by intracarotid artery hyperosmolar mannitol induces sterile inflammatory and innate immune responses. Proc. Natl. Acad. Sci. 118: e2021915118.
Chu, C, et al., (2018) Real-Time MRI Guidance for Reproducible Hyperosmolar Opening of the Blood-Brain Barrier in Mice. Front. Neurol. 9:921.
Chu, C, et al., (2020) Optimization of osmotic blood-brain barrier opening to enable intravital microscopy studies on drug delivery in mouse cortex. J. Controlled Release 317:312-321.
Chu, C, et al., (2022) Hyperosmolar blood-brain barrier opening using intra-arterial injection of hyperosmotic mannitol in mice under real-time MRI guidance. Nat. Protoc. 17:76-94.
Erdlenbruch B, et al., (2003) Alkylglycerol opening of the blood-brain barrier to small and large fluorescence markers in normal and C6 glioma-bearing rats and isolated rat brain capillaries. Br J Pharmacol. 140 (7): 1201-10.
Gan Y, et al., (2013) Gene delivery with viral vectors for cerebrovascular diseases. Front Biosci (Elite Ed). 5:188-203. Review.
Gao, Y, et al., (2021) Imaging as a tool to accelerate the translation of extracellular vesicle-based therapies for central nervous system diseases. WIREs Nanomedicine Nanobiotechnology 13: e1688.
Guillaume D J, et al. (2010) Intra-arterial chemotherapy with osmotic blood-brain barrier disruption for aggressive oligodendroglial tumors: results of a phase I study. Neurosurgery. 66 (1): 48-58.
Haluska, M, et al., (2004) Osmotic Blood-Brain Barrier Modification for the Treatment of Malignant Brain Tumors. Clin. J. Oncol. Nurs. 8:263-267.
Ikeda, M, et al., (2002) Synergistic effect of cold mannitol and Na (+)/Ca (2+) exchange blocker on blood-brain barrier opening. Biochem. Biophys. Res. Commun. 291:669-674.
Janowski, M, et al., (2016) Walczak, P. & Pearl, M. S. Predicting and optimizing the territory of blood-brain barrier opening by superselective intra-arterial cerebral infusion under dynamic susceptibility contrast MRI guidance. J. Cereb. Blood Flow Metab. 36:569-575.
Kroll, R A, et al., (1998) Outwitting the Blood-Brain Barrier for Therapeutic Purposes: Osmotic Opening and Other Means. Neurosurgery 42:1083-1099.
Lesniak, W G, et al., (2019) A Distinct Advantage to Intraarterial Delivery of 89 Zr-Bevacizumab in PET Imaging of Mice With and Without Osmotic Opening of the Blood-Brain Barrier. J. Nucl. Med. 60:617-622.
Lesniak, W G, et al. (2019a) PET imaging of distinct brain uptake of a nanobody and similarly-sized PAMAM dendrimers after intra-arterial administration. Eur. J. Nucl. Med. Mol. Imaging 46:1940-1951.
Lochhead, J J, et al., (2020) Structure, Function, and Regulation of the Blood-Brain Barrier Tight Junction in Central Nervous System Disorders. Front. Physiol. 11:914.
Marchi, N, et al., (2007) Seizure-Promoting Effect of Blood?Brain Barrier Disruption. Epilepsia 48:732-742.
Nhan T, et al. (2013) Drug delivery to the brain by focused ultrasound induced blood-brain barrier disruption: Quantitative evaluation of enhanced permeability of cerebral vasculature using two-photon microscopy. J Control Release. 172 (1): 274-280.
Neuwelt et al., (1979) Osmotic Blood-brain Barrier Disruption: A New Means of Increasing Chemotherapeutic Agent Delivery. Trans Am. Neurol. Assoc. 104:256-260.
Pandit, R, et al., (2020) The blood-brain barrier: Physiology and strategies for drug delivery. Adv. Drug Deliv. Rev. 165-166:1-14.
Pardridge, W. M. (2002) Drug and gene delivery to the brain: the vascular route, Neuron. 36:555-558.
Preston E, et al., (2008) Graded reversible opening of the rat blood-brain barrier by intracarotid infusion of sodium caprate. J Neurosci Methods. 168 (2): 443-9.
Rapoport, SI (2001) Advances in osmotic opening of the blood-brain barrier to enhance CNS chemotherapy. Expert Opin. Investig. Drugs 10:1809-1818.
Siegal, T, et al., (2000) In vivo assessment of the window of barrier opening after osmotic blood-brain barrier disruption in humans. J. Neurosurg. 92:599-605.
Van Vliet, E A, et al., (2007) Blood-brain barrier leakage may lead to progression of temporal lobe epilepsy. Brain 130, 521-534.
Vosjan, M J, et al., (2010) Conjugation and radiolabeling of monoclonal antibodies with zirconium-89 for PET imaging using the bifunctional chelate p-isothiocyanatobenzyl-desferrioxamine. Nat. Protoc. 5:739-743.
Whelan, R, et al., (2021) Hargaden, G. C. & Knox, A. J. S. Modulating the Blood-Brain Barrier: A Comprehensive Review. Pharmaceutics 13:1980.
Zawadzki, M, et al., (2019) Real-time MRI guidance for intra-arterial drug delivery in a patient with a brain tumor: technical note. BMJ Case Rep. 12: e014469.
Zawadzki, M, et al., (2021) Follow-up of intra-arterial delivery of bevacizumab for treatment of butterfly glioblastoma in patient with first-in-human, real-time MRI-guided intra-arterial neurointervention. J. NeuroInterventional Surg. 13:1037-1039.

What is claimed is:

1. A method of delivering a substantially improved fraction of at least one therapeutic agent, at least one contrast agent, or both, to an organ of a subject, said method comprising:
administering an effective amount of an osmotically active composition to the subject to disrupt a blood-organ barrier at said organ; and
administering a therapeutically effective amount of a formulation comprising at least one therapeutic agent, at least one contrast agent, or both to the subject,
wherein the osmotically active composition induces transient permeabilization of the blood-organ barrier to enhance the delivery of the at least one therapeutic agent, at least one contrast agent, or both, to the organ of the subject.

2. The method of claim 1, wherein the osmotically active composition is administered intraarterially.

3. The method of claim 1, wherein the formulation is administered intraarterially.

4. The method of claim 3, wherein the formulation is administered intraarterially after blood-organ barrier disruption has been detected.

5. The method of claim 1, further comprising ceasing the administration of the osmotically active composition to the subject before intraarterially administering the formulation.

6. The method of claim 5, wherein the cessation of the administration of the osmotically active composition to the subject is for a time x, wherein x is in a range from about 1 sec to about 30 min.

7. The method of claim 1, wherein the osmotically active composition and the formulation are both administered intraarterially via the same or a different catheter.

8. The method of claim 1, wherein the organ is selected from a brain, skin, salivary glands, tongue, and jaw muscle, and any other organ of the body.

9. The method of claim 8, wherein the formulation is administered intraarterially via a catheter placed in an artery that is feeding the organ or a portion thereof.

10. The method of claim 9, wherein the formulation displaces blood in the artery increasing extravasation of the at least one therapeutic agent, at least one contrast agent, or both, to the organ.

11. The method of claim 1, wherein the osmotically active composition comprises at least one osmotically active species.

12. The method of claim 1, wherein the osmotically active composition comprises at least one osmotically active agent, at least one ionic osmotically active agent, and water.

13. The method of claim 12, wherein the blood-organ barrier is the blood-brain barrier (BBB) and the osmolality of the osmotically active composition is greater than 1,372 mOsm/L.

14. The method of claim 12, wherein the at least one osmotically active agent comprises mannitol, dextrose, propylene glycol, glycerol, sorbitol, trehalose, erythritol, or any combination thereof.

15. The method of claim 12, wherein the at least one ionic osmotically active agent comprises CaCl₂), KBr, KCl, LiCl, NaCl, NaBr, Na₂SO₄, or any combination thereof.

16. The method of claim 12, wherein the blood-organ barrier is not the BBB and the osmolality of the osmotically active composition is greater than 312 mOsm/L.

17. The method of claim 1, wherein the formulation comprising the at least one therapeutic agent, at least one contrast agent, or both, is a sterile aqueous or non-aqueous solution, suspension, or emulsion.

18. The method of claim 17, wherein the formulation further comprises at least one pharmaceutically acceptable excipient selected from the group consisting of additional osmotically active substances, preservatives, antimicrobial or antibacterial agents, anti-oxidants, chelating agents, electrolytes, diluents, buffering agents, surfactants, and inert gases.

19. The method of claim 1, wherein the at least one therapeutic agent comprises: a neurologically active agent that acts at synaptic and neuroeffector junction sites; a species that acts on the central nervous system and/or the peripheral nervous system; an anti-cancer agent or drug; an antibody or an immunoconjugate comprising an antibody; or a small molecule therapeutic agent.

20. A method of treating cancer, an inflammatory disease, a genetic disease, a neurological disease, or any other disease in a subject, said method comprising:
administering an effective amount of an osmotically active composition to the subject to disrupt a blood-organ barrier at an organ to be treated; and
administering a therapeutically effective amount of a formulation comprising at least one therapeutic agent to the subject,
wherein the osmotically active composition induces transient permeabilization of the blood-organ barrier to enhance delivery of the at least one therapeutic agent to an organ of the subject, thereby treating said disease.
